(19) United States
(12) Patent Application Publication
(10) Pub. No.: US 2005/0148003 A1
(43) Pub. Date: Jul. 7, 2005

(54) Methods of correcting a luminescence value, and methods of determining a corrected analyte concentration

(76) Inventors: Steven Keith, Chapel Hill, NC (US); Phyllis J. Palmer, Durham, NC (US)

Correspondence Address: DAVID W. HIGHET;BECTON, DICKINSON AND COMPANY, 1 BECTON DRIVE, MC110, FRANKLIN LAKES, NJ, 07417, US

(21) Appl. No.: 11/002,157

(22) Filed: Dec. 2, 2004

Related U.S. Application Data
(63) Continuation-in-part of application No. 10/967,220, filed on Oct. 19, 2004.
Application No. 10/967,220 is a continuation-in-part of application No. 10/721,797, filed on Nov. 26, 2003.

(51) Int. Cl. C12Q 1/68; G01N 33/53; G01K 11/00; G06F 19/00; G01N 33/48; G01N 33/50

(52) U.S. Cl. 435/6; 435/7.1; 702/19; 374/162

(57) ABSTRACT

The invention is directed to methods of correcting a luminescence value, and methods of determining a corrected analyte concentration, by use of a device capable of providing a signal when a binding protein binds to at least one analyte, and a thermometer. The invention is also directed to systems which include such a device, and a processor for correcting measured luminescence of a reporter group based on a measured temperature. The invention is further directed to apparatuses that include a memory for storing luminescence information and temperature information, and a processor for correcting luminescence information. The invention is further directed to computer programs for executing the methods of the invention, and machine-readable storage medium on which programs are recorded.

### CROSS REFERENCE TO RELATED APPLICATIONS

The present invention is a continuation-in-part of co-pending U.S. patent application Ser. No. 10/967,220, filed on Oct. 19, 2004, which is a continuation-in-part of U.S. patent application Ser. No. 10/721,797, both of which applications are incorporated herein by reference in their entirety.

### FIELD OF THE INVENTION

The present invention is directed to methods of correcting a luminescence value, and/or methods of determining a corrected analyte concentration, by use of a device having at least one binding protein having at least one reporter group attached thereto, and a thermometer. The present invention is also directed to systems that include such devices and a processor for correcting measured luminescence of a reporter group based on a measured temperature. The present invention is further directed to computer programs for executing the methods of the invention, and computer or machine-readable storage medium on which programs are recorded. Further included are apparatuses that include a memory for storing luminescence information of a reporter group and temperature information, and a processor for correcting the luminescence information.

### BACKGROUND OF THE INVENTION

Monitoring in vivo concentrations of physiologically relevant analytes, such as glucose, lactate or oxygen, in certain individuals is vitally important to patients' health, as it may improve diagnosis and treatment of various diseases and disorders. For example, high or low levels of glucose or other analytes may have detrimental effects. The monitoring of glucose is particularly important to individuals with diabetes, as diabetics must determine when insulin is needed to reduce glucose levels in their bodies or when additional glucose is needed to raise the level of glucose in their bodies.

Currently, many diabetics use the “finger stick” method to monitor their blood glucose levels. Patient compliance using this method is problematic due to pain caused by frequent (several times per day) sticks. As a consequence, there have been efforts to develop non-invasive or minimally invasive in vivo and more efficient in vitro methods for frequent and/or continuous monitoring of blood glucose or other glucose-containing biological fluids. Some of the most promising of these methods involve the use of a biosensor.

Biosensors are devices capable of providing specific quantitative or semi-quantitative analytical information using a biological recognition element that is combined with a transducing (detecting) element. The biological recognition element of a biosensor determines the selectivity, so that only the target analyte or analytes to be measured leads to a signal. The transducer translates the recognition of the biological recognition element into a semi-quantitative or quantitative signal.

The approaches to frequent and/or continuous in vivo monitoring tend to fall into two general categories: “non-invasive” and “minimally invasive.” Noninvasive monitoring determines analyte levels by directly tracking spectroscopic changes in skin and tissue. Infrared radiation and radio wave impedance spectroscopy are examples of this technology. Progress with these approaches has been slow due to the requirement for frequent calibration, reproducible sample illumination, and variances in spectroscopic backgrounds between individuals. The “minimally invasive” approach avoids direct extraction of blood from the body and relies on the monitoring of signal changes in biological fluids using an intermediate sensing element. Biosensors of this type are devices capable of providing specific quantitative or semi-quantitative analytical information using a biological recognition element that is combined with a transducing (detecting) element.

Most conventional systems for frequent or continuous analyte monitoring involve amperometric biosensors that employ enzymes such as glucose oxidase (GOx) to oxidize glucose to glucuronic acid and hydrogen peroxide, generating an electrochemical signal. These sensors are subject to inaccurate measurement due to oxygen deficiency and buildup of oxidation by-products. An accurate measurement of glucose concentrations requires an excess of oxygen, which is generally not present in human blood or interstitial fluid. Also, the electrochemical reaction itself generates a buildup of oxidation byproducts that may inhibit and degrade both the enzyme and its protective layer.

Biosensors based on optical rather than electrochemical signals have also been developed and may offer significant improvements in stability and calibration. For example, referencing an analyte-dependent optical signal against a second analyte-independent signal can correct for sources of noise and instability in the sensor. Current optical sensing methods rely on enzymatic chemistry such as glucose oxidase. In one common method, an oxygen-sensitive fluorescent dye is used to monitor the consumption of oxygen by the GOx enzymatic reaction. Although this is an optical biosensor, with the fluorescence signal level varying with changing oxygen levels, such a sensor is subject to the same problems as amperometric devices based on this same chemistry: oxygen deficiency and enzyme degradation.

To overcome the challenges associated with enzyme sensing (e.g., GOx), whether electrochemical or optical, non-enzymatic protein-based optical or fluorescent sensing is being explored. Labeled concanavalin A and dextran have been used to create a competitive FRET assay; however, this system requires entrapment of both components, and the dynamic range of the assay is limited. See, Ballerstadt, R., Schultz, J. S.; “Competitive-binding assay method based on fluorescence quenching of ligands held in close proximity by a multivalent receptor.” Anal. Chem. Acta 345 (1-3): 203-212 (1997). See also, Russell, R. J., Pishko M. V., Gefrides C. C., McShane, M. J., Cote, G. L.; “A fluorescence-based glucose biosensor using concanavalin A and dextran encapsulated in a poly(ethylene glycol) hydrogel” Anal. Chem. 71 (15): 3126-3132 (1999).

Another protein-based sensing chemistry uses the Esherichia coli (E. coli) periplasmic receptor, glucose-galactose binding protein (GGBP) to generate a fluorescence signal in response to glucose binding. See, for example, Tolosa, L., I. Gryczynski, L. R. Eichhorn, J. D. Dattelbaum, F. N. Castellano, G. Rao, and J. R. Lakowicz; “Glucose sensor for low-cost lifetime-based sensing using a genetically engineered protein” Anal. Biochem. 267: 114-120 (1999); Hellinga, H. W., and J. S. Marvin; “Protein engineering and the development of generic biosensors.” Trends Biotechnol. 16: 183-189 (1998); Salins, L. L., R. A. Ware, C. M. Ensor, and S. Daunert, “A novel reagentless sensing system for measuring glucose based on the galactose/glucose-binding protein” Anal Biochem 294: 19-26 (2001); and de Lorimier, R. M., J. J. Smith, M. A. Dwyer, L. L. Looger, K. M. Sali, C. D. Paavola, S. S. Rizk, S. Sadigov, D. W. Conrad, L. Loew, and H. W. Hellinga. “Construction of a fluorescent biosensor family” Protein Sci. 11: 2655-2675 (2002). GGBP undergoes a substantial conformation change upon ligand binding, trapping the ligand between its two globular domains. See, for example, Shilton, B. H., M. M. Flocco, M. Nilsson, and S. L. Mowbray; “Conformational changes of three periplasmic receptors for bacterial chemotaxis and transport: the maltose-, glucose/galactose- and ribosebinding proteins” J. Mol. Biol. 264: 350-363 (1996). By site-specifically labeling the protein with an environmentally sensitive fluorophore this attribute can be exploited to generate a fluorescent signal. See, for example, Salins, L. L., R. A. Ware, C. M. Ensor, and S. Daunert; “A novel reagentless sensing system for measuring glucose based on the galactose/glucose-binding protein” Anal Biochem 294: 19-26 (2001). Because GGBP neither consumes glucose nor generates reaction products, it can be used as a reagentless sensor. This may provide greater accuracy and reliability than amperometric biosensors.

A functional frequent and/or continuous biosensor must couple the sensing element to the optical sensing elements while maintaining sensor integrity and functionality as well as patient comfort. For example, the biological recognition element and accompanying transducing element should preferably be incorporated within biocompatible material that shields the sensing element from the immune system, permits analyte diffusion in and out, and avoids leaching of the sensing element into the patient blood or other biological fluid (e.g., interstitial fluid). Because binding proteins require orientational control and conformational freedom to enable effective use, many physical absorption and random or bulk covalent surface attachment or immobilization strategies as taught in the literature generally are either suboptimal or unsuccessful. Further, a means for interrogating the sample with light in a reproducible and/or controlled fashion must be devised.

One approach is to couple the sensing element to one end of an optical fiber and to couple the optical elements such as excitation sources or detectors to the other end. However, coupling of binding proteins to one end of an optical fiber is subject to the above-mentioned challenge of preserving conformational and/or orientational mobility of the protein. In addition, fiber optic cabling is often impractical from a patient-use point of view since patients may need to remove or replace the sensor periodically. Replacement of the entire fiber can be costly and inconvenient. Finally, the optical system, comprising, e.g., excitation sources, detectors, and other optical elements must be sufficiently robust to tolerate or correct for changes in optical alignment due, for example, to patient motion or drift of the electronics in the optical reader. The optical system must also be sufficiently sensitive to detect signal from reporter dyes without relying on high power consumption and/or large-sized elements that would render the system unportable and hence unwearable.

### SUMMARY OF THE INVENTION

The invention is directed to methods of correcting a luminescence value of a reporter group, and methods of determining a corrected analyte concentration, by use of a device having at least one binding protein having at least one reporter group thereto, and a thermometer. The reporter group may be capable of providing a luminescent signal when a binding protein binds to at least one analyte.

The invention is also directed to devices themselves, systems that include such devices, and processors for correcting measured luminescence of a reporter group based on a measured temperature. The invention is further directed to apparatuses that include a memory for storing luminescence information and temperature information, and a processor for correcting luminescence information. The invention is further directed to computer programs for executing the methods of the invention, and machine-readable storage medium on which programs are recorded.

The aspects, advantages and other features of the invention will become apparent in view of the following detailed description, which discloses various non-limiting embodiments of the invention.

### BRIEF DESCRIPTION OF THE DRAWINGS

The invention will be more readily understood with reference to the embodiments thereof illustrated in the attached figures, in which:

FIG. 1 depicts sample data from an experiment in which optical sensors were placed in scintillation vials containing glucose solutions, which were located in a heating block.

FIG. 2 depicts fluorescence data plotted as a function of temperature from the high concentration (30 mM) portion of an exemplary experiment.

FIG. 3 depicts fluorescence data plotted as a function of temperature from the low concentration (5 mM) portion of an exemplary experiment.

FIG. 4 depicts the overall and high-temperature sensitivities for tested sensors.

FIG. 5 depicts uncorrected and corrected sensor signals for a cooling cycle according to an exemplary experiment.

### DETAILED DESCRIPTION OF THE INVENTION

Embodiments of the invention will now be described. The following detailed description of the invention is not intended to be illustrative of all embodiments. In describing embodiments of the present invention, specific terminology is employed for the sake of clarity. However, the invention is not intended to be limited to the specific terminology so selected. It is to be understood that each specific element includes all technical equivalents that operate in a similar manner to accomplish a similar purpose.

The present invention involves at least one binding-protein engineered to bind at least one analyte of interest within a desired clinical or analytical range. In addition, one or more luminescent reporter groups are associated with the binding protein. The one or more binding proteins along with their associated reporter groups comprise the sensing element.

The sensing element, comprising one or more binding proteins, one or more reporter groups, and optional reference groups, may be immobilized at the end of the optical conduit or inside a disposable tip that interfaces with the optical conduit. Immobilization of the sensing element in the optical conduit or inside the disposable tip may be accomplished by depositing a thin layer of the sensing element, for example, by dip or spin coating, covalent attachment, plasma treatment, and the like directly onto the optical conduit or tip. Alternately, the sensing element can be first immobilized in a polymeric matrix and the matrix then attached to the optical conduit, or tip either by adhesives, injection molding, dip or spin coating, plasma coating, vacuum deposition, ink jet technology, covalent, ionic, or van der Waals interactions, by mechanical attachment or any combination thereof. In an alternate embodiment, a thin layer of sensing chemistry may be attached to the optical conduit and then covered with a semi-permeable membrane.

The optical system is capable of interrogating the luminescent response of the reporter and reference groups by passing light from an electromagnetic excitation source down the optical conduit to the distal end containing the sensing element. The optical system may also monitor and interpret the return signals generated by the luminescence response of the reporter group and reference group. The luminescent properties of the reporter group, either wavelength, intensity, lifetime, energy transfer efficiency, or polarization, change in response to analyte binding or unbinding from the binding protein.

The present invention is directed to utilizing the temperature impact on luminescence to determine a corrected luminescence value from at least one reporter group, which in turn may be used to determine and/or correct analyte concentration.

Many properties of luminescent labels can change in response to temperature changes, including for example, intensity, wavelength of maximum emission, lifetime, FRET efficiency (Fluorescence Resonance Energy Transfer), polarization, and other properties known to those skilled in the art.

The present inventors have found that luminescence intensity, such as fluorescence activity, is inversely and non-linearly related to temperature through several possible mechanisms. Mechanisms producing temperature dependence in luminescence include, but are not limited to, solvent interactions, loss of energy through increased molecular motion, etc.

This temperature effect on luminescence can be used in multiple ways. By way of non-limiting example, first, by measuring fluorescence of a biologically inert substrate, changes in temperature can be calculated. Second, measuring the temperature in a biological sample (e.g., near the tip of an in vivo fluorescence biosensor) allows correction of a luminescent signal. Third, a combination device can be constructed wherein luminescence measurements are used td measure temperature and the temperature measurement is used to correct another luminescent measurement.

The luminescence-temperature relation of a dye, e.g., fluorescein, NBD, Texas Red, etc. is non-linear over a wide range of temperatures (e.g., from room temperature to human body temperature). The relationship is approximately linear, however, over small temperature ranges (e.g., over the range of body temperatures normally experienced). Methods are described herein to take advantage of localized linearity when correcting for temperature.

For certain luminescence-based applications, precise temperature correction is not required. For example, if fluorescence changes due to temperature are on the order of 2-3% per degree C., then typical room temperature oscillations might result in fluorescence changes that are not as significant relative to other sources of experimental error, as temperature changes of a greater degree.

In biological applications, e.g., in vivo biosensors, it would be useful to compensate for various temperature effects. For example, not only are in vivo temperatures different from room temperature where reference readings might be taken, but body temperatures may vary by several degrees depending on many factors. Additionally, in the case of e.g., biosensors located in or under the skin, external sources of temperature variation (sunlight, air conditioning, clothing, etc.) may result in an even wider temperature range.

Further, for certain applications of luminescence measurement, e.g., measuring glucose via fluorescently labeled binding proteins, errors in luminescence measurement may be magnified in the sensor output. For example, errors of fluorescence on the order of 3% may result in errors in reported glucose by up to 12-15% depending on performance characteristics of the protein-dye. Errors of 12-15% may be unacceptable for commercial applications of this type biosensor. Therefore, a temperature shift of 1-2%, if unaccounted for, could render some luminescent biosensors unviable.

The inverse relationship between luminescence (e.g., fluorescence) and temperature is due to an increase of molecular motion with increasing temperature, which results in more molecular collisions and subsequent loss of energy. Fluorescence is an energy dissipation phenomenon, and any competing routes of energy loss will reduce fluorescence.

The temperature-luminescence relationship can be utilized using steady-state luminescence measurements as described herein. The methods can also be applied to time-domain luminescence measurements. Temperature affects how quickly luminescence decays over time. Changes in the decay curves of a luminescing device, such as a biosensor can be used to calculate temperature, or temperature measurements can be used to correct time-domain data.

Devices

The present invention encompasses devices that include at least one binding protein having at least one reporter group attached thereto, and a thermometer. The thermometer may be capable of measuring a temperature of a biological sample in proximity to the at least one binding protein. The at least one reporter group may be capable of providing a detectable signal when said binding protein binds to at least one analyte as defined herein. (The term “analyte” is also referred to herein as a “target analyte” or “ligand.” The singular use of any of these terms is meant to encompass at least one of the same or different analytes.) The term “reporter group” refers to one or more of the same or different reporter groups as defined herein. (The singular use of the term “reporter group” is meant to encompass at least one of the same or different reporter groups.) The term “binding protein” refers to one or more of the same or different binding proteins, polypeptides and/or mutated binding proteins as defined herein. (The term “binding protein” is meant to encompass at least one of the same or different binding proteins.) These definitions, as with all of the definitions throughout this application, may relate to any of the embodiments of the inventions described herein, and are not limited to any particular embodiment(s) by virtue of their location within the specification, or otherwise.

Devices in accordance with the present invention include both in vivo and ex vivo devices, including for example, biosensors. Biosensors in accordance with the present invention may take any form apparent to those skilled in the art, including for example, a matrix as described e.g., in U.S. patent application Ser. No. 10/039,833 filed Jan. 4, 2002, and Ser. No. 10/776,643 filed Feb. 12, 2004, which are hereby incorporated by reference.

For example, devices in accordance with the present invention may include (i) an optical conduit having a proximal end and a distal end; and (ii) a sensing element in optical proximity to the distal end of the optical conduit that comprises at least one binding protein that is adapted to bind with at least one target analyte; said sensing element also comprising at least one reporter group.

The optical conduit, which may vary in length from approximately 0.1 cm to 1 meter, couples light into and out of an optical system and into and out of the sensing element. For example, the optical conduit may be a lens, a reflective channel, a needle, or an optical fiber. The optical fiber may be either a single strand of optical fiber (single or multimode) or a bundle of more than one fiber. In one embodiment, the bundle of fibers is bifurcated. The fiber may be non-tapered or tapered so that it can penetrate the skin of a patient.

An optical system may be connected to the proximal end of the optical conduit. The optical system consists of a combination of one or more excitation sources and one or more detectors. It may also consist of filters, dichroic elements, a power supply, and electronics for signal detection and modulation. The optical system may optionally include a microprocessor.

The optical system interrogates the sample either continuously or intermittently by coupling one or more interrogating wavelengths of light into the optical conduit. The one or more interrogating wavelengths then pass through the optical conduit and illuminate the sensing element. A change in analyte concentration results in a change of the wavelength, intensity, lifetime, energy transfer efficiency, and/or polarization of the luminescence of the reporter group, which is a part of the sensing element. The resulting changed luminescence signal passes back through the optical conduit to the optical system where it is detected, interpreted, and stored and/or displayed. In certain embodiments, the optical system comprises multiple excitation sources. One or more of these sources may be modulated to permit dynamic signal processing of the detected signal, thereby enhancing signal-to-noise and detection sensitivity. Modulation may also be used to reduce power consumption by the device or to increase the lifetime of the sensing element by minimizing undesirable phenomena such as photo-bleaching. The optical system can also include one or more electromagnetic energy detectors that can be used for detecting the luminescence signal from the reporter and optional reference groups as well as for internal referencing and/or calibration. The overall power consumption of the optical system is kept small to permit the device to be operated using battery power.

The sensing element comprises one or more binding proteins that are adapted to bind with at least one target analyte, and at least one reporter group. A sensing or biological recognition element of a sensor determines selectivity, so that the analyte(s) to be measured lead to a signal. The selection may be based for example, on biochemical recognition of the analyte(s), where the chemical structure of the analyte (e.g., glucose) is unchanged, or biocatalysis in which the element catalyzes a biochemical reaction of the analyte. Thus, the term “binding protein(s)” refers to protein(s) (including mutated proteins) that interact with specific analytes in a manner capable of providing or transducing a detectable and/or reversible signal differentiable either from when analyte is not present, analyte is present in varying concentrations over time, or in a concentration-dependent manner. The transduction event includes continuous, programmed, and episodic means, including one-time or reusable applications. Reversible signal transduction may be instantaneous or may be time-dependent providing a correlation with the presence or concentration of analyte is established. Binding proteins mutated in such a manner to effect transduction may be used in accordance with the present invention.

The suitable binding protein may be any one or more of those described in co-pending, commonly owned U.S. Patent Application Publication No. 2003/0153026; U.S. Patent Application Publication No. 2003/0134346; U.S. Patent Application Publication No. 2003/0130167; and U.S. patent application Ser. No. 10/721,091 for “Compositions and Methods for Measuring Analyte Concentrations” to Terry Amiss, et al. filed on Nov. 26, 2003, the contents of which are incorporated herein by reference in their entirety. Suitable binding proteins may also be any one of those described in U.S. Pat. No. 6,277,627, U.S. Pat. No. 6,197,534, or WO 03/060464 A2 the entire contents of which are incorporated herein by reference in their entirety.

As used herein and as set forth in the applications incorporated herein by reference, the term “mutated binding protein” (for example “mutated Galactose/Glucose Binding Protein” (“GGBP”)) includes mutated binding proteins from bacteria containing an amino acid(s) that has been substituted for, deleted from, or added to the amino acid(s) present in naturally occurring protein. Possible substitutions, deletions or insertions may involve fewer than 5 amino acid residues, or one or two residues. Exemplary mutations of binding proteins include the addition or substitution of cysteine groups, non-naturally occurring amino acids (see e.g., Turcatti, et at. J Bio, Chem. 1996 271, 33, 19991-19998, which is incorporated herein by reference) and replacement of substantially non-reactive amino acids with reactive amino acids to provide for the covalent attachment of electrochemical or photo-responsive reporter groups. By “reactive amino acid” is meant an amino acid that can be modified with a labeling agent analogous to the labeling of cysteine with a thiol reactive dye. Non-reactive amino acids include alanine, leucine, phenylalanine, and others, which possess side chains which cannot be readily modified once incorporated in a protein (see Greg T. Hermanson, Bioconjugate Techniques, Academic Press, 1996, San Diego, pp. 4-16 for classification of amino acid side chain reactivity).

Mutated binding protein may be engineered to have a histidine tag on the proteins N-terminus, C-terminus, or both. Histidine fusion proteins are widely used in the molecular biology field to aid in the purification of proteins. Exemplary tagging systems produce proteins with a tag containing about six histidines and preferably such tagging does not compromise the binding activity of the mutated binding protein. According to certain embodiments of the present invention, the binding protein may have one, two, three or more mutations. For example, according to certain embodiments of the present invention, the mutated binding protein is glucose galactose binding protein (GGBP) having a triple mutation including a cysteine substituted for an glutamic acid at position 149, an arginine substituted for an alanine at position 213 and a serine substituted for leucine at position 238 (E149C/A213R/L238S). The protein may be labeled at the 149 position with e.g., N,N′-dimethyl-N-(iodoacetyl)-N′-(7-nitrobenz-2-oxa-1,3-diazol-4-yl)ethylenediamine (IANBD amide)oxy. This mutated GGBP (E149C/A213R/L238S) is specific for glucose, and the reporter group undergoes a fluorescence intensity change in response to glucose binding.

Further examples of mutated binding proteins that may be used in accordance with the present invention are set forth, e.g., in U.S. patent application Ser. No. 10/039,833 filed on Jan. 4, 2002 and U.S. Ser. No. 10/776,643 filed Feb. 12, 2004, which are incorporated by reference herein.

In order to accurately determine glucose concentration in a biological sample such as blood, saliva, tears, sweat, urine, cerebral spinal fluid, lymph fluid, interstitial fluids, plasma, serum, ocular solutions, animal tissue and media, etc., it may be desirable to adjust the binding constant of the sensing molecule of a biosensor so as to match the physiological and/or pathological operating range of the biological solution of interest. Without the appropriate binding constant, a signal may be out of range for a particular physiological and/or pathological concentration. Additionally, sensors may be configured using more than one protein, each with a different binding constant, to provide accurate measurements over a wide range of glucose concentrations as discussed in U.S. Pat. No. 6,197,534, which is hereby incorporated herein by reference.

A transducer translates the recognition of the biological recognition element into a semi-quantitative or quantitative signal. Possible transducer technologies are optical, electrochemical, acoustical/mechanical or colorimetrical. The optical properties that have been exploited include absorbance, fluorescence/phosphorescence, bio/chemiluminescence, reflectance, light scattering and refractive index. As described further below, conventional reporter groups such as fluorescent compounds or other luminescent groups may be used in accordance with the present invention. It is contemplated, however, that other reporter groups besides luminescent labels may be used.

“Reporter groups” in accordance with the present invention may be capable of undergoing a luminescence change upon binding of the binding protein to the target analyte. Specific mutations of sites and/or attachment of certain reporter groups may act to modify a binding constant in an unpredictable way. Additionally, a binding protein containing reporter groups may have a desirable binding constant, but not result in an easily detectable signal change upon analyte binding. Some of the factors that determine sensitivity of a particular reporter probe attached to a particular protein for the detection of a specific analyte, include, but are not limited to, the nature of the specific interactions between the selected probe and amino acid residues of the protein.

“Detectable signal change,” as used herein, refers to the ability to recognize a change in a property of a reporter group in a manner that enables the detection of ligand-protein binding. For example, the binding proteins (or mutated binding proteins) may include a detectable reporter group whose detectable characteristics alter upon a change in protein conformation that occurs on glucose binding.

Examples of reporter groups include, but are not limited to, organic dyes, pairs of organic dyes, fluorescent or bioluminescent fusion proteins, pairs of fluorescent or bioluminescent fusion proteins, or any combination of the above. The reporter group may consist of a donor and acceptor undergoing fluorescence resonance energy transfer. Other luminescent labeling moieties include lanthanides such as europium (Eu3+) and terbium (Tb3+), as well as metal-ligand complexes, including those of ruthenium [Ru (II)], rhenium [Re(I)], or osmium [Os (II)], typically in complexes with diimine ligands such as phenanthrolines.

According to certain embodiments, the at least one reporter group may include a luminescent label and may have luminescent properties. Luminescent reporter groups include but are not limited to, organic aromatic dye molecules covalently coupled to cysteine residues in the protein or, for example, luminescent biomolecules such as proteins fused to the engineered binding protein. Cysteine or other amino acid groups may be engineered into the binding protein to provide sites of attachment for the luminescent reporter molecule. Binding of an analyte to the binding protein results in a change in the luminescent properties of one or more reporter groups. The luminescent property affected may be the absorption or emission wavelength, absorption or emission intensity, emission lifetime, emission polarization, and/or energy transfer efficiency. Binding of the analyte is also reversible, with the unbinding resulting again in a change in the luminescent properties of the reporter molecule.

The terms “luminescent” and “luminescence” include, but are not limited to fluorescence, phosphorescence, bioluminescence, electrochemical and chemiluminescence and any other form of luminescence as would be apparent to one of ordinary skill in the art. Accordingly, the luminescent label may be for example, a fluorescent label, a phosphorescent label, or other luminescent label. By way of example, fluorescent labels that may be excited to fluoresce by exposure to certain wavelengths of light may be used in accordance with the present invention. Phosphorescently labeled binding proteins may also be used in accordance with the present invention.

According to certain embodiments, the at least one reporter group may include a fluorophore. As used herein, “fluorophore” refers to a molecule that absorbs energy and then emits light. Non-limiting examples of fluorophores useful as reporter groups in this invention include fluorescein, coumarins, rhodamines, 5-TMRIA (tetramethylrhodamine-5-iodoacetamide), Quantum Red™, Texas Red™, Cy3, N-((2-iodoacetoxy)ethyl)-N-methyl)amino-7-nitrobenzoxadiazole (IANBD), 6-acryloyl-2-dimethylaminonaphthalene (acrylodan), pyrene, Lucifer Yellow, Cy5, Dapoxyl® (2-bromoacetamidoe-thyl)sulfonamide, (N-(4,4-difluoro-1,3,5,7-tetramethyl-4-bora-3a,4a-diaza-s-indacene-2-yl)iodoacetamide (Bodipy507/545 IA), N-(4,4-difluoro-5,7-diphenyl-4-bora-3a,4a-diaza-s-indacene-3-propionyl)-N′-iodoacetylethylenediamine (BODIPY® 530/550 IA) 5-((((2-iodoacetyl)amino)ethyl) amino)naphthalene-1-sulfonic acid (1,5-IAEDANS), and carboxy-X-rhodamine, 5/6-iodoacetamide (XRIA 5,6). Preferably, IANBD is used. Many detectable intrinsic properties of a fluorophore reporter group may be monitored to detect glucose binding. Some properties that can exhibit changes upon glucose binding include fluorescence lifetime, fluorescence intensity, fluorescence anisotropy or polarization, and spectral shifts of fluorescence emission. Changes in these fluorophore properties may be induced from changes in the fluorophore environment such as those resulting from changes in protein conformation. Environment-sensitive dyes such as IANBD are particularly useful in this respect. Other changes of fluorophore properties may result from interactions with the analyte itself or from interactions with a second reporter group, for example when FRET (fluorescence resonance energy transfer) is used to monitor changes in distance between two fluorophores.

According to certain embodiments, fluorophores that operate at long excitation and emission wavelengths (for example, about 600 nm or greater excitation or emission wavelengths) may be used when the molecular sensor is to be used in vivo, for example, incorporated into an implantable biosensor device (the skin being opaque below 600 nm). Thiol-reactive derivatives of Cy-5 can be prepared, for example, as taught by H. J. Gruber, et al, Bioconjugate Chem., (2000), 11, 161-166, which is incorporated herein by reference. Conjugates containing these fluorophores, for example, attached at various cysteine mutants constructed in mutated GGBPs, can be screened to identify those that result in the largest change in fluorescence upon glucose binding.

According to certain embodiments, the reporter group is a luminescent label that results in a mutated protein with an affinity for glucose producing a detectable shift in luminescence characteristics on glucose binding. The change in the detectable characteristics may be due to an alteration in the environment of the label, which is bound to the mutated protein.

Labeled mutated binding proteins having fluorophore reporter probes may be used in accordance with the present invention, according e.g., to the procedure set forth by Cass et al., Anal. Chenz. 1994, 66, 3840-3847, or as otherwise described and known to those skilled in the art.

Examples of further suitable reporter groups in accordance with the present invention are set forth, for example, in U.S. Ser. No. 10/039,833 filed on Jan. 4, 2002, U.S. Ser. No. 10/721,021 filed Nov. 26, 2003, and U.S. Ser. No. 10/776,643 filed Feb. 12, 2004, which are incorporated by reference herein.

The reporter group may be “attached to” or “associated with” the protein or mutated protein by any conventional means known in the art. As used herein, the terms “attached to” and “associated with” are used interchangeably to mean that the reporter group is covalently or non-covalently associated with the binding protein such that upon binding of a target analyte to the binding protein, there is a change in the reporter group's luminescence properties such as wavelength, intensity, lifetime, energy transfer efficiency, and/or polarization.

For example, the reporter group may be attached via amines or carboxyl residues on the protein. By way of example, covalent coupling via thiol groups on cysteine residues may be used. For example, for mutated GGBP, cysteines located at position 11, position 14, position 19, position 43, position 74, position 107, position 110, position 112, position 113, position 137, position 149, position 152, position 213, position 216, position 238, position 287, and position 292 are preferred in the present invention. Any thiol-reactive group known in the art may be used for attaching reporter groups such as fluorophores to a cysteine of an engineered protein.

For example, an iodoacetamide bromoacetamide, or maleimide are well known thiol-reactive moieties that may be used for this purpose. Attachment methods are also described for example, in U.S. Ser. No. 10/039,833 filed on Jan. 4, 2002, which is herein incorporated by reference.

Optionally, the sensing element may also contain one or more reference groups. Unlike the reporter group, the reference group has a luminescence signal that is substantially unchanged upon binding of the target analyte to the binding protein. The luminescence signal from the reference group provides an internal optical standard that can be used to correct for optical artifacts due to for example, electronic drift in the optical system or to motion of the sample or optical conduit. The reference group can also be used for calibration. The reference group can be attached to any number of components of a device including the sensing element, a binding protein not containing the reporter group, a polymer matrix, a polymer chain, a biomolecule that is not a binding protein, an optical conduit, or a tip. In certain embodiments, the reference group is attached to a binding protein that has been engineered to show little or no significant response to the analyte at physiologically relevant concentrations.

According to certain embodiments, the sensing element is in optical proximity to the optical conduit. “Optical proximity” means that components of the device are close enough to one another such that an optical signal can be transmitted to or received from one object by another. The sensing element may be placed in optical proximity to the optical conduit in a number of ways, for example: attached directly to the optical conduit; attached to a connector that is attached to the optical conduit; attached to a polymer chain or a polymer matrix that is attached to the optical conduit; or attached to a polymer chain or a polymer matrix that is attached to a connector that is attached to the optical conduit. The sensing element may be permanently affixed to the optical conduit or replaceably attached such that the sensing element can be replaced conveniently and economically.

In other embodiments, the sensing element may further comprise one or more reference groups. Unlike the reporter group, the reference group has a luminescence signal that may be substantially unchanged upon binding of the target analyte to the binding protein. “Substantially unchanged” means the luminescence change of the reference group is significantly less than the luminescence change undergone by the reporter group. The reference group, which can consist of luminescent dyes and/or proteins, is used for internal referencing and calibration. The reference group can be attached to any number of components of the device including the sensing element, a binding protein not containing the reporter group, the polymer matrix, the polymer chain, a biomolecule that is not a binding protein, the optical conduit, or a tip.

The sensing element (typically this refers to the binding protein with the associated reporter group and optional reference group) may be attached directly to the distal end of the optical conduit using for example covalent, ionic, or van der Waals interactions, dip coating, spin coating, plasma coating, or vacuum deposition. The sensing element may also be attached to a connector, which allows the sensing element to be readily detachable so that it becomes replaceable.

In other embodiments, the sensing element may be attached to or immobilized in a polymeric matrix. As used herein, the term “matrix” may be any two dimensional or three-dimensional structure that is permeable to an analyte. The matrix may optionally prevent substantial interference from other biomolecules and may be substantially biocompatible. In certain embodiments, the matrix allows the binding protein to retain some degree of conformational and/or orientational mobility. The matrix may consist of multiple layers, with an inner layer serving to retain the binding protein, and one or more outer layers to control the permeability and/or achieve biocompatibility. For example, the polymer matrix may be any one of those described in co-pending, commonly owned U.S. application Ser. No. 10/428,295, filed May 2, 2003, the entire contents of which are incorporated herein by reference. The immobilization may be accomplished for example, by covalently linking the sensing element to the polymer matrix or by physically entrapping the sensing element within the matrix. In the instance where the polymer matrix physically entraps the sensing element, the matrix pores are sized to retain the sensing element. In embodiments where the sensing element is attached to the polymeric matrix, the sensing element is attached to the matrix using, for example, covalent or ionic linkage. The polymer matrix can be attached to the distal end of the optical conduit using adhesives, dip or spin coating, plasma coating, covalent, ionic, or van der Waals interactions, a mechanical connector or combinations thereof.

In other embodiments, the sensing element is attached to a polymeric chain. The method of attaching the sensing element to the polymeric chain includes, but is not limited to, covalent, ionic, and van der Waals interactions and combinations thereof. The polymer chain is attached to the distal end of the optical conduit using, for example, dip or spin coating, plasma coating, vacuum deposition, covalent, ionic, or van der Waals interactions, or combinations thereof.

In other embodiments, the device may further include a tip (either tapered or non-tapered) that is designed to pierce the skin to allow the sensing element to contact body fluids in the intradermal or subcutaneous space. The tip may be disposable. The tip may be made of plastic, steel, glass, polymer, or any combination of these or similar materials. The tip may be attached directly to the optical conduit (fiber) using adhesives or a mechanical fitting. The tip may also be used to house the optical conduit containing the sensing element, such that it encases the optical conduit and sensing element. In certain embodiments, the sensing element may be contained within the tip.

The device may further comprise a connector that may be used to attach the components of the device to one another. The connector may be, for example, any mechanical device, such as standard fiber optic connectors, luer locks, plastic, metal, or glass sleeves, or spring-loaded housings. For instance, the connector may be used to attach the sensing element to the optical conduit, or to attach the optical conduit to the optical system. The primary purpose of the connector is to provide a component that allows the other components to be readily detachable so that the component becomes replaceable.

According to certain embodiments, light of one or more wavelengths produced in the optical system may be channeled down an optical conduit to the sensing element. The optical conduit may be either an optical fiber or a short light guide that transmits light with minimal loss. The sensing element may include one or more binding proteins with one or more associated luminescent reporter groups either immobilized in a polymeric matrix, attached to a polymer chain, incorporated in a disposable tip, attached directly to the distal end of the optical conduit, or attached to a connector. The sensing element may include additional luminescent reference groups that are optionally attached to biomolecules, polymers, or organic molecules for the purpose of providing a reference or calibration signal. A sensing element can be attached to the distal end of an optical conduit, either directly or via a polymer matrix, or attached to a disposable tip that is attached to the distal end of the optical conduit. In this case, the disposable tip is positioned against the optical conduit either mechanically, via adhesive, or by any other suitable means known to those of skill in the art.

According to certain embodiments, a dichroic mirror or beamsplitter may be used to direct light from an electromagnetic energy source to the optical conduit. Excitation sources may consist of, but are not limited to, for example arc lamps, laser diodes, or LEDs. In these embodiments, the optical conduit may be for example, a fiber optic cable, and the same fiber may be used to transmit excitation light from electromagnetic energy source to the sensing element and also to transmit the luminescence signals from the reporter or reference groups back to the optical system. A dichroic element may separate the return signal from the excitation light and directs the signal to electromagnetic energy detectors. Detectors may include, but are not limited to, for example, photodiodes, CCD chips, or photomultiplier tubes. In the event that multiple luminescent signals are returned from the sensing element, additional dichroic elements may be used to direct portions of the return signals to multiple detectors. Preferably, a luminescent reference group that is analyte insensitive is included along with the analyte-dependent reporter molecule to provide a reference signal. This reference signal can be used, for example, to correct for optical or electronic drift.

According to other embodiments in which a bifurcated optical bundle or fused optical fiber arrangement may be used to transmit light to and from the sensing element, light from an excitation source may be transmitted down one arm of the bifurcated fiber bundle. Return luminescent signals from the sensing element may be detected using the second arm of the bifurcated fiber, so that in this case the fiber bundling serves to separate excitation from return luminescence. Dichroic optics, beamsplitters, or polarizers may additionally be used to further divide the return luminescence, based for example on wavelength or polarization. Optionally, bandpass filters can be used to select the luminescent wavelength to be detected. A power supply supplies power to the optical system.

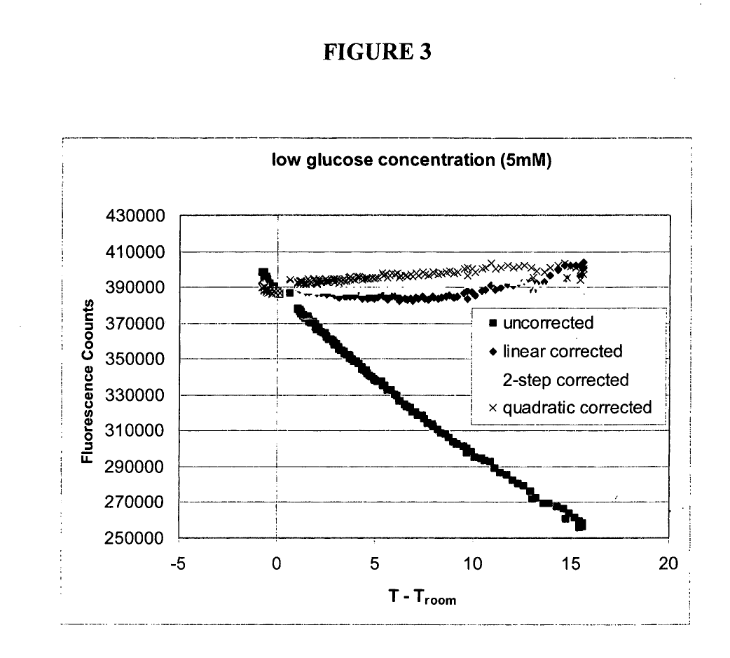

Various methods or means of attaching the sensing element to the end of an optical conduit may be used, when, for example, the optical conduit comprises an optical fiber. One skilled in the art will recognize that attention must be given to design considerations such as obtaining sufficient or intimate contact between the sensing element and the optical fiber, preventing delamination of the sensing element from the optical fiber in operation to ensure that light is efficiently transmitted to and from the sensing element. Furthermore, maintaining the integrity of the sensing element during operation is important to ensure that a reliable signal response may be obtained. For example, when used in vivo sensing element may be subject to various environments which may cause shrinkage, swelling, deterioration, or negatively impact other desirable functional characteristics including signal intensity, luminescence, response time, etc. Thus, optimal attachment methods or means may vary depending on the characteristics, configuration, and dimensions of the particular sensing element or particular application. For example, the attachment methods shown in FIG. 3 of U.S. Ser. No. 10/967,220, to which this application claims priority, may be used either individually or in combination.

According to certain embodiments, the sensing element may be attached directly to the distal end of the optical fiber using for example covalent, ionic, or van der Waals interactions, dip coating, spin coating, plasma coating, vacuum deposition, ink jet technology, or combinations thereof. Alternatively the sensing element, comprising the binding proteins, associated reporter groups, and optional reference groups, can be attached to a polymer, such as for example a monolayer or long chain polymer, and the polymer attached directly to the distal end of the optical fiber using for example, dip or spin coating, plasma coating, vacuum deposition, covalent, ionic, or van der Waals interactions, ink jet technology, or combinations thereof.

According to other embodiments an optical fiber is within the inside of a needle (fiber-in-needle). As used herein, the term “needle” includes but is not limited to a micro-needle. The needle may have a modified distal end such as a bevel to control piercing depth and/or one or more side ports to permit access of the analyte to the sensing element 6 contained in needle. The sensing element may be positioned inside the needle such that it may be attached directly to optical fiber using any of the methods described herein and/or in any of the applications incorporated herein by reference, or, alternatively, may have only mechanical contact with optical fiber. In alternate embodiments, the distal end of needle may be crimped to mechanically fix the sensing element to the needle.

In certain embodiments, the external diameter of the optical fiber is between about 50-400 microns, preferably between about 50-200 microns and the internal diameter of the needle is dimensioned slightly larger than the external diameter to accommodate the insertion of the optical fiber into the needle. In a variation, the needle may be mechanically fixed to optical fiber by, for instance, friction fit or crimping needle onto the optical fiber. In alternate embodiments, the optical fiber may be chemically fixated inside the needle by glue or any other suitable means known to those skilled in the art. In this regard, a biosensor tip assembly including a needle with an integrated optical fiber and sensing element may be manufactured to be disposable for episodic use or may remain in vivo for continual use. In other embodiments, the optical fiber may be removably insertable into and out of the needle such that the needle may remain in vivo and the optical fiber may be inserted and removed as desired for episodic use. In certain embodiments, the proximal end of the needle includes an optical coupling member configured and dimensioned to receive an attachable optical component thereto for instance to connect or interface to an optical system. In certain embodiments the needle is a straight needle, although in alternate embodiments the needle may have one or more bends or bending portions anywhere along its length. Furthermore, in other alternative embodiments, the distal end of needle may include a bent tip portion at the distal end extending distally beyond and adjacent to matrix and may include a reflective or light scattering surface or layer with the light reflecting surface facing the optical fiber to improve luminescence and/or amplify the return signal. Other embodiments of a needle assembly include one or more ports or holes through which the analyte may flow or migrate to permit access of the analyte to the sensing element contained in the needle.

Other embodiments include a wearable optical biosensor. In certain embodiments, the tip body comprise a steel needle approximately 1-10 mm in length containing within it the sensing element immobilized or fixed onto an optical fiber. The fiber, sensing element, and needle are positioned in a mount. The tip body or needle, containing the optical fiber and the sensing element, may be inserted perpendicularly into the skin of a patient so that the sensing element resides in either the intradermal or subcutaneous space. In an exemplary embodiment, the needle is fixedly mounted on a mount such that a controlled insertion depth may be obtained. In this regard, the needle may extend into the skin of a patient a distance between about 0.1 mm to about 10 mm, or between about 1 mm to about 2 mm. An adhesive ring may hold the mount plus needle assembly in place. The optical system may then clamp over the mount plus needle assembly, with the connector interfacing the optical fiber with the optical system. The optical reader can also be separated from the platform by, for instance, approximately 0.02-1 meter and connected to the rest of the system with an optical fiber. Excitation sources may consist of, but are not limited to, for example arc lamps, laser diodes, or LEDs. Detectors may consist of, but are not limited to, for example, photodiodes, CCD chips, or photomultiplier tubes. In an alternative embodiment, a plurality of tip bodies or needle assemblies may be attached to a single mount. In this regard the tip bodies or needle assemblies may be configured to test multiple analytes wherein each needle assembly is configured to test a single analyte. In other embodiments, tip bodies or needle assemblies may be attached to the mount such that a drug may be delivered through at least one tip body or needle assembly. Thus, a drug delivery system may be designed such that a proper dosage of drug may be calculated based upon the testing of an analyte and delivered via a tip body or needle assembly attached to the same biosensor mount. In these embodiments, the tip body or needle assembly used for drug delivery may comprise one or more ports to deliver the drug therethrough.

In other embodiments, a thermometer such as a temperature probe may be contained within, adjacent to, or attached to at least one tip body or needle assembly. A temperature probe could be, for example, a thermocouple or an optical temperature monitor using, for example, a temperature sensitive fluorophore. In other variations, the biosensor tip can be incorporated into a wearable patch device, wherein the proximal end of the tip body is attached to a patch and the patch is configured and dimensioned to be worn on the exterior skin of the patient. In another embodiment, the biosensor tip may be incorporated into a watch, wherein the proximal end of the tip body is attached to a watch and the watch is configured and dimensioned to be worn on the exterior wrist area of the patient.

The term “thermometer” is used herein to include any device or composition capable of measuring temperature. Thermometers in accordance with the present invention include, but are not limited to, all forms of temperature sensors including for example, thermocouples and/or infrared devices. Thermometers may also include, for example, luminescent dyes capable of detecting and/or measuring temperature. Accordingly, in accordance with certain embodiments of the invention, the at least one reporter group and the thermometer may be the same luminescent dye, or may be separate dyes used in conjunction with one another.

Certain thermometers in accordance with the present invention may be capable of detecting and/or measuring a temperature of a biological sample, or at least a portion of a biological sample, in proximity to the at least one binding protein. “In proximity to the at least one binding protein” may mean for example, that the thermometer may measure the temperature of a biological sample from about 0 to 6 inches, or about 1 to 3 inches away from the at least one binding protein, however, proximity is not limited to these distances and the thermometer may in fact be farther from the binding protein and still be encompassed by the present invention. According to certain embodiments of the invention, the temperature is accurate to at least about 0.4° C., or to at least about 0.2° C.

The term “biological sample” is intended to include all in vivo and in vitro biological sample(s), including, but not limited to semi-liquid and liquid samples such as blood, saliva, tears, sweat, urine, cerebral spinal fluid, lymph fluid, interstitial fluids, plasma, serum, ocular solutions, animal tissue and media, and any other biological sample(s) known to those in the art.

The term “analyte” or “target analyte” encompasses one or more of the same or different analytes that can be detectable using binding protein. According to certain embodiments, the analyte to be detected includes glucose and the analyte presence or concentration to be determined is that of glucose. Numerous other analytes and analyte concentrations may be detected and determined in accordance with the present invention, however. The target analytes can be any molecule or compound where the concentration is desired to be measured.

Examples of classes of analytes that can be measured include, but are not limited to amino acids, peptides, polypeptides, proteins, carbohydrates, lipids, nucleotides, oligonucleotides, polynucleotides, glycoproteins or proteoglycans, lipoproteins, lipopolysaccharides, drugs, drug metabolites, small organic molecules, inorganic molecules, natural polymers, and synthetic polymers.

As used herein, “carbohydrate” includes, but is not limited to monosaccharides, disaccharides, oligosaccharides and polysaccharides. “Carbohydrate” also includes, but is not limited to, molecules comprising carbon, hydrogen and oxygen that do not fall within the traditional definition of a saccharide—i.e., an aldehyde or ketone derivative of a straight chain polyhydroxyl alcohol, containing at least three carbon atoms. Thus, for example, a carbohydrate may contain fewer than three carbon atoms.

As used herein, the term “lipid” is used it is in the art, i.e., substances of biological origin that are made up primarily or exclusively of nonpolar chemical groups such that they are readily soluble in most organic solvents, but only sparingly soluble in aqueous solvents. Examples of lipids include, but are not limited to, fatty acids, triacylglycerols, glycerophospholipids, sphingolipids, cholesterol, steroids and derivatives thereof. For example, “lipids” include but are not limited to, the ceramides, which are derivatives of sphingolipids and derivatives of ceramides, such as sphingomyelins, cerebrosides and gangliosides. “Lipids” also include, but are not limited to, the common classes of glycerophospholipds (or phospholipids), such as phosphatidic acid, phosphatidylethanolamine, phosphatidylcholine, phosphatidylserine, phosphatidylinositol, phosphatidylglycerol and the like.

As used herein, a “drug” can be a known drug or a drug candidate, whose activity or effects on a particular cell type are not yet known. A “drug metabolite” is any of the by-products or the breakdown products of a drug that is changed chemically into another compound or compounds. As used herein, “small organic molecule” includes, but is not limited to, an organic molecule or compound that does not fit precisely into other classifications highlighted herein.

In certain embodiments, all of the target analytes are of the same class of compounds, e.g., proteins, or fatty acids or carbohydrates. According to other embodiments, at least one of the target analytes is in a different compound class from the other target analytes. For instance, the device can measure a protein or polypeptide and a carbohydrate or carbohydrates. In yet other embodiments of the present invention, none of the target analytes is in the same class of compounds. Furthermore, the target analytes may be specific compounds within a class of compounds, e.g., glucose, palmitate, stearate, oleate, linoleate, linolenate, and arachidonate. Alternatively, the target analytes may be an entire class of compounds, or a portion or subclass thereof, e.g., fatty acids. Specific examples of target analytes include, but are not limited to, glucose, galactose, fatty acids, lactic acid (lactate), C-reactive protein, carbohydrates, and anti-inflammatory mediators, such as cytokines, eicosanoids, or leukotrienes. In certain embodiments, the target analytes are fatty acids, C-reactive protein, and leukotrienes. In other embodiments, the target analytes are glucose, lactic acid and fatty acids.

“Fatty acids,” as used herein include all fatty acids, including free fatty acids (FFA) and fatty acids esterified to other molecules. Examples of specific fatty acids include, but are not limited to, palmitate, stearate, oleate, linoleate, linolenate, and arachidonate. The term “free fatty acid” is used herein as it is in the art in that FFA are not part of other molecules such as triglycerides or phospholipids. Free fatty acids also include non-esterified fatty acids that are bound to or adsorbed onto albumin. As used herein, the term “unbound free fatty acid” (unbound FFA) is used to denote a free fatty acid or free fatty acids that are not bound or adsorbed onto albumin or other serum proteins. In fact, it is believed that unbound FFA circulate in low levels in the body. (See McArthur M. J., et al., J. Lipid Res., 40: 1371-1383, (1999), the entirety of which is hereby incorporated by reference.) Furthermore, there is also evidence that an equilibrium between albumin-bound free fatty acids and unbound free fatty across cell membranes exists and is readily established. For example, unbound FFA can diffuse across from an adipose cell onto albumin, where the FFA is transported to other tissues. The albumin-bound FFA then diffuses across the cell membrane of another cell where the FFA can be stored or used as an energy source. (See Abreu, M. S. C., et al., Biophys. J., 84: 386-399, (2003), and Weisiger, R. A., Am. J. Physiol-Gastr., 277: G109-G119, (1999), the entireties of which are hereby incorporated by reference.)

Further analytes in accordance with the present invention are set forth in one or more of the following: U.S. Provisional application 60/577,931 filed on Jun. 9, 2004, U.S. Ser. No. 10/039,833 filed Jan. 4, 2002, U.S. Ser. No. 10/721,021 filed Nov. 26, 2003 and/or U.S. Ser. No. 10/776,643 filed Feb. 12, 2004, which are incorporated herein by reference.

In certain embodiments, the target analytes are not labeled. While not limited to such, the devices of the present invention may be particularly useful in an in vivo setting for measuring target analytes as they occur or appear in a subject. As such, the target analytes need not be labeled. Of course, unlabeled target analytes may also be measured in an in vitro or in situ setting as well. In other embodiments, the target analytes may be labeled. Labeled target analytes can be measured in an in vivo, in vitro or in situ setting.

The selection of reporter groups and/or binding proteins may be affected by which analyte concentration is to be determined. Appropriate reporter groups and/or binding proteins may be selected by one of ordinary skill in the art based on the analyte concentration to be determined and based on the present disclosure and the disclosures of U.S. Provisional application 60/577,931 filed on Jun. 9, 2004, U.S. Ser. No. 10/039,833 filed Jan. 4, 2002, U.S. Ser. No. 10/721,021 filed Nov. 26, 2003 and/or U.S. Ser. No. 10/776,643 filed Feb. 12, 2004, which are incorporated herein by reference. Additionally, as would be apparent to one skilled in the art in view of this disclosure, the parameter values of the formulae set forth herein may vary based on the analyte concentration to be determined, the reporter groups, and/or the binding proteins. Such modifications and variations are also encompassed by the present invention.

The binding proteins having at least one reporter group attached thereto may be able to generate a signal. In certain embodiments of the present invention, the devices, systems or methods of the present invention may further include one or more signal detectors or other means for acquiring the signal information or for measuring the signal. The generated signal may be indicative of the binding of the analytes to binding domains, and thus, indicative of the concentration of the analytes. In other words, the binding of analytes to the binding domain either creates or alters the quality of a signal that is discernable using a detector. Changes in signal quality include, but are not limited to, light wavelength shift and signal intensity. In certain embodiments, the binding domains do not generate a signal when not bound to the target analytes. In other embodiments, the binding domains generate a signal, even when not bound to a target analyte, but the binding of the target analyte, however, still changes the quality of the signal, such that binding is discernable. It is also possible that the binding of the analyte to a binding domain may cause a decrease in signal intensity, simply provided that the alteration in the signal is discernable to the detector.

In certain embodiments of the current invention, the detector is a fluorometer that can measure the wavelength and/or intensity of fluorescent light. Examples of other detectors can be an infrared spectrophotometer, a UV-Vis spectrophotometer, a photodiode that can be used in surface plasmon resonance (SPR) protocols and even the naked eye. In SPR, the refractive index properties of a sample near a surface will change when the target molecule is present, and the intensity of the reflected light is dampened by the presence of a metal surface at the interface of the sample and glass media. The decrease in intensity occurs at a well-defined angle, which is dependent on the refractive
indices of the two media, referred to as the “resonance angle.”

The devices of the current invention can be used in a variety of settings, including in vivo, in vitro and in situ. In certain embodiments of the present invention, the devices are medical devices or implants. When the implants are used in an in vivo setting, the implants should be biocompatible such that they produce little or no detectable inflammation/rejection reaction. Certain embodiments for rendering the implants more biocompatible comprises coating the implants with biocompatible polymers, such as poly(urethane) elastomers, poly(urea) and poly(vinylchloride). Poly(urethane) elastomers posses excellent mechanical properties including high tensile strength, good tear and abrasion resistance and a relatively good stability in biological environments. The excellent mechanical properties of segmented polyurethanes are attributed to their two phase morphology derived from microphase separation of soft and hard segments. When polyurethanes are used for long term medical implants, the soft segments are typically formed from a poly(ether) macrodiol such as poly(tetramethylene oxide) (PTMO), whereas the hard segments are derived from a diisocyanate such as 4,4′-methylenediphenyl diisocyanate (MDI) and a diol chain extender such as 1,4-butanediol. Other coatings of the implant may include poly(urea) compositions disclosed in U.S. Pat. No. 6,642,015, which is hereby incorporated by reference.

Other formulations for rendering the implant biocompatible are disclosed in U.S. Pat. No. 6,706,532, which is hereby incorporated by reference. Additionally, Quinn et al., (Biomaterials, 18: 1665-1670 (1997)), which is herein incorporated by reference, reports an amperometric glucose electrode biosensor constructed with poly(ethylene glycol) (PEG) hydrogels as an outer layer to provide biocompatibility for enzymatic biosensors.

According to certain embodiments the devices of the present invention are sensor devices (such as biosensors) that include an optical sensor and a thermometer capable of measuring a temperature of a biological sample in proximity to the optical sensor. The optical sensor may be capable of measuring at least one analyte in a biological sample in proximity to the optical sensor. A non-limiting example of an optical sensor in accordance with the present invention includes a fluorescent optical oxygen probe, such as that marketed by Ocean Optics (FOXY Fiber Optic Oxygen Sensors).

Further devices in accordance with the present invention include devices having at least one binding protein having at least one reporter group attached thereto, where the at least one reporter group is capable of detecting a temperature of a biological sample in proximity to the at least one binding protein. Thus, according to these embodiments, a reporter group may serve as both an analyte detector and temperature sensor or at least two reporter groups are utilized in which at least one reporter group is capable of detecting a temperature of a biological sample in proximity to the at least one binding protein.

Alternatively, each binding protein-reporter group pair may serve as either the analyte sensor or the temperature sensor, rather than serving as both. Devices according to the present invention may include at least one binding protein-reporter group pair that serves as an analyte sensor and at least one pair that serves as a temperature sensor. Further embodiments of the devices of the invention may include one or more additional analyte-sensing compounds, and/or one or more additional thermometers, such as temperature sensors. According to certain embodiments, the same type of dye may be attached to different proteins, where one protein is active and another is not.

In the embodiments of the present invention, the sensing element or manufactured tip device may be sterile. In this regard, “sterile” means essentially free of microorganisms or bacteria. In certain methods of manufacture, the assembled components may be sterilized periodically after each step of manufacture. For example, in certain embodiments, the sleeve may be sterilized after each step of manufacture ultimately ending in an aseptically packaged device. Alternatively, the assembled fiber and sensing element or manufactured tip device can be sterilized in a terminal step.

Methods

Methods in accordance with the present invention may include acquiring luminescence information of at least one reporter group, acquiring temperature information of a biological sample, and determining a corrected luminescence value based on the temperature information, where the corrected luminescence value is indicative of a concentration of at least one analyte in the sample. The method may further include determining a concentration of the at least one analyte based on the corrected luminescence information. According to certain embodiments, luminescence information comprises information acquired from at least one binding protein having the at least one reporter group attached thereto, wherein the at least one reporter group luminesces when the at least one binding protein binds to at least one analyte. According to further embodiments, the temperature information includes information regarding the temperature of at least a portion of the biological sample in proximity to the at least one binding protein.

According to certain methods of the invention, corrected luminescence at room temperature (L_{RT}) may be a function of at least one of the following variables: luminescence (L(T)) of a reporter group at temperature (T); temperature (T); and room temperature (T_{R}). According to certain methods, luminescence of a reporter group at room temperature (L_{RT}) may be determined by the following formula:
L_{RT}=L(T)/[1+SQ1*(T−T_{R})²+SQ2*(T−T_{R})]
wherein L_{RT} is luminescence corrected to room temperature, L(T) is luminescence of a reporter group at a temperature (T), T_{R} is room temperature, SQ1 is a coefficient relating to the magnitude of the quadratic relationship between temperature and luminescence, and SQ2 is a coefficient relating to the magnitude of the linear relationship between temperature and luminescence.

As used herein, the term “room temperature” refers to a temperature at which reference luminescence values might be obtained during, e.g., factory calibration of a sensor or sensors representative of a manufacturing lot of sensors. The term “room temperature” usually refers to a temperature between about 21 and 23 degrees Celsius. Insofar as reference luminescence readings may be taken at temperatures outside that range, “room temperature” may refer to any such temperature.

The present invention also encompasses methods of correcting luminescence information emitted from a sensor, wherein luminescence at room temperature (L_{RT}) is determined by the following formula:
L_{RT}=L(T)/[1+SQ1*(T−T_{R})²+SQ2*(T−T_{R})]
wherein the variables are as set forth above.

Such methods include determining luminescence at room temperature (L_{RT}) by one or more of the following: determining luminescence (L(T)) of a reporter group at a temperature (T); determining room temperature (T_{R}); determining a coefficient (SQ1) relating to the magnitude of the quadratic relationship between temperature and luminescence, and determining a coefficient (SQ2) relating to the magnitude of the linear relationship between temperature and luminescence.

As indicated above, in accordance with the methods of the invention, the term “luminescence” may include fluorescence, phosphorescence or any other type of luminescence known to those skilled in the art. Therefore, according to certain embodiments of the invention, fluorescence corrected to room temperature (F_{RT}) may be a function of at least one of the following variables: fluorescence (F(T)) of a reporter group at temperature (T); temperature (T); and room temperature (T_{R}). According to certain methods, fluorescence at room temperature (F_{RT}) may be determined by the following formula:
F_{RT}=F(T)/[1+SQ1*(T−T_{R})²+SQ2*(T−T_{R})]
wherein F_{RT} is fluorescence corrected to room temperature, F(T) is fluorescence of a reporter group at a temperature (T), T_{R} is room temperature, SQ1 is a coefficient relating to the magnitude of the quadratic relationship between temperature and fluorescence, and SQ2 is a coefficient relating to the magnitude of the linear relationship between temperature and fluorescence.

The present invention also encompasses methods of correcting fluorescence information emitted from a sensor, wherein fluorescence at room temperature (F_{RT}) is determined by the following formula:
F_{RT}=F(T)/[1+SQ1*(T−T_{R})²+SQ2*(T−T_{R})]
wherein the variables are as set forth above.

Such methods include determining fluorescence at room temperature (F_{RT}) by one or more of the following: determining fluorescence (F(T)) of a reporter group at a temperature (T); determining room temperature (T_{R}); determining a coefficient (SQ1) relating to the magnitude of the quadratic relationship between temperature and fluorescence, and determining a coefficient (SQ2) relating to the magnitude of the linear relationship between temperature and fluorescence.

According to certain embodiments of the invention, corrected luminescence (L_{RT}) may be a function of at least one of the following variables: luminescence (L(T)) of a reporter group at a temperature (T); temperature (T); temperature sensitivity (S1) around a skin or body temperature region; and sensitivity (S2) in a region between room temperature (T_{R}) and skin temperature (T_{S}). Thus, according to certain embodiments, corrected luminescence is determined by the following formula:
L_{RT}=L(T)/[1+S1*(T−T_{S})]/[1+S2*(T_{S}−T_{R})]
wherein L_{RT} is luminescence corrected to room temperature, L(T) is luminescence of a reporter group at a temperature (T), S1 is temperature sensitivity around a skin or body temperature region, and S2 is a sensitivity applying to a region between room temperature (T_{R}) and skin temperature (T_{S}). Temperature sensitivity (S) is expressed as a fraction of the luminescence signal lost (from the correct or reference signal) per degree.

Thus, the present invention also encompasses methods of correcting luminescence information emitted from a sensor, wherein corrected luminescence at room temperature (L_{RT}) is determined by the following formula:
L_{RT}=L(T)/[1+S1*(T−T_{S})]/[1+S2*(T_{S}−T_{R})]
wherein the variables are as set forth above.

These methods may include determining luminescence at room temperature (L_{RT}) by one or more of the following: determining luminescence (L(T)) of a reporter group at a temperature (T); determining temperature sensitivity (S1) around a skin or body temperature region; and determining a sensitivity (S2) applying to a region between room temperature (T_{R}) and skin temperature (T_{S}).

Again noting that “luminescence” may include fluorescence, phosphorescence or other types of luminescence, corrected fluorescence (F_{RT}) may be a function of at least one of the following variables: fluorescence (F(T)) of a reporter group at a temperature (T); temperature (T); temperature sensitivity (S1) around a skin or body temperature region; and sensitivity (S2) in a region between room temperature (T_{R}) and skin temperature (T_{S}). Thus, according to certain embodiments, corrected luminescence is corrected fluorescence determined by the following formula:
F_{RT}=F(T)/[1+S1*(T−T_{S})]/[1+S2*(T_{S}−T_{R})]
wherein F_{RT} is fluorescence corrected to room temperature, F(T) is fluorescence of a reporter group at a temperature (T), S1 is temperature sensitivity around a skin or body temperature region, and S2 is a sensitivity applying to a region between room temperature (T_{R}) and skin temperature (T_{S}).

Thus, the present invention also encompasses methods of correcting fluorescence information emitted from a sensor, wherein corrected fluorescence at room temperature (F_{RT}) is determined by the following formula:
F_{RT}=F(T)/[1+S1*(T−T_{S})]/[1+S2*(T_{S}−T_{R})]
wherein the variables are as set forth above.

These methods may include determining fluorescence at room temperature (F_{RT}) by one or more of the following: determining fluorescence (F(T)) of a reporter group at a temperature (T); determining temperature sensitivity (S1) around a skin or body temperature region; and determining a sensitivity (S2) applying to a region between room temperature (T_{R}) and skin temperature (T_{R}).

According to other embodiments, corrected luminescence at room temperature (L_{RT}) may be a function of at least one of the following variables: luminescence of a reporter group (L(T)) at a temperature (T); temperature (T); and temperature sensitivity (S1) around a skin or body temperature region (T_{S}). Thus, according to certain embodiments, corrected luminescence is determined by the following formula:
L_{RT}=L(T)*FC/[1+S1*(T−T_{S})]
wherein L_{RT} is luminescence corrected to room temperature; L(T) is luminescence of a reporter group at a temperature (T), S1 is temperature sensitivity around a skin or body temperature region (T_{S}); and FC is a correction factor which accounts for the change in luminescence due to the difference in the nominal temperature of skin of a mammal for which analyte concentration is being determined (“T-skin”), and room temperature (“T-room”).

Thus, the present invention also encompasses methods of correcting luminescence information emitted from a sensor, wherein corrected luminescence is determined by the following formula:
L_{RT}=L(T)*FC/[1+S1*(T−T_{S})].

These methods may include determining luminescence at room temperature (L_{RT}) by one or more of the following: determining luminescence (L(T)) of a reporter group at a temperature (T); and determining temperature sensitivity (S1) around a skin or body temperature region (T_{S}), where FC is a correction factor which accounts for the change in luminescence due to the temperature difference between nominal T-skin and T-room.

According to other embodiments where luminescence is fluorescence, the corrected fluorescence at room temperature (F_{RT}) may be a function of at least one of the following variables: fluorescence (F(T)) of a reporter group at a temperature (T); temperature (T); and temperature sensitivity (S1) around a skin or body temperature region (T_{S}). Thus, according to certain embodiments, corrected luminescence is corrected fluorescence determined by the following formula:
F_{RT}=F(T)*FC/[1+S1*(T−T_{S})]
wherein F_{RT} is fluorescence correct to room temperature; F(T) is fluorescence of a reporter group at a temperature (T), S1 is temperature sensitivity around a skin or body temperature region (T_{S}); and FC is a correction factor which accounts for the change in luminescence due to the temperature difference between nominal T-skin and T-room.

Thus, the present invention also encompasses methods of correcting fluorescence information emitted from a sensor, wherein corrected fluorescence is determined by the following formula:
F_{RT}=F(T)*FC/[1+S1*(T−T_{S})].

These methods may include determining fluorescence at room temperature (F_{RT}) by one or more of the following: determining fluorescence (F(T)) of a reporter group at a temperature (T); and determining temperature sensitivity (S1) around a skin or body temperature region (T_{S}), where FC is a correction factor which accounts for the the change in luminescence temperature difference between nominal T-skin and T-room.

The present invention also encompasses other reformulations or approximations of the equations presented herein, including the equations presented in the examples, as would be apparent to those skilled in the art upon review of this disclosure.

The present invention further encompasses methods including converting a reference luminescence (e.g., fluorescence) of a reporter group at a reference temperature (T) into a luminescence at a reference body temperature. Thereafter, an actual measured luminescence may be converted to the reference body temperature, based on the relationship between luminescence and the reference body temperature. Such a conversion may be performed, for example, by one or more of the following equations:
L(T)=L_{RT}*[1+SQ1*(T−T_{R})²+SQ2*(T−T_{R})],
L(T)=L_{RT}*[1+S1*(T−T_{S})]*[1+S2*(T_{S}−T_{R})], or
L(T)=L_{RT}*[1+S1*(T−T_{S})]/FC,
wherein L(T) is luminescence of a reporter group at a temperature (T), L_{RT} is luminescence at room temperature, SQ1 is a coefficient relating to the magnitude of the quadratic relationship between temperature and luminescence, SQ2 is a coefficient relating to the magnitude of the linear relationship between temperature and luminescence, T_{R} is room temperature, S1 is temperature sensitivity around a skin or body temperature region (T_{S}), S2 is a sensitivity applying to a region between room temperature (T_{R}) and skin temperature, and FC is a correction factor which accounts for T-skin, T-body, and T-room.

Thus, the present invention also encompasses methods of correcting luminescence information emitted from a sensor, wherein corrected luminescence is determined by any of the following formulae:
L(T)=L_{RT}*[1+SQ1*(T−T_{R})²+SQ2*(T−T_{R})],
L(T)=L_{RT}*[1+S1*(T−T_{S})]*[1+S2*(T_{S}−T_{R})], or
L(T))=L_{RT}*[1+S1*(T−T_{S})]/FC,
wherein L(T) is luminescence of a reporter group at a temperature (T), L_{RT} is luminescence at room temperature, SQ1 is a coefficient relating to the magnitude of the quadratic relationship between temperature and luminescence, SQ2 is a coefficient relating to the magnitude of the linear relationship between temperature and luminescence, T_{R} is room temperature, S1 is temperature sensitivity around a skin or body temperature region (T_{S}), S2 is a sensitivity applying to a region between room temperature (T_{R}) and skin temperature, and FC is a correction factor which accounts for T-skin, T-body, and T-room. Such methods include measuring and/or determining any of the above variables and solving for L(T) and/or L_{RT} using one or more of the above formulae.

Thus, where luminescence is fluorescence, methods of the present invention include converting a reference fluorescence of a reporter group at a reference temperature (T) into fluorescence at a reference body temperature. Thereafter, an actual measured fluorescence may be converted to the reference body temperature, based on the relationship between fluorescence and the reference body temperature. Such a conversion may be performed, for example, by one or more of the following equations:
F(T)=F_{RT}*[1+SQ1*(T−T_{R})²+SQ2*(T−T_{R})],
F(T)=F_{RT}*[1+S1*(T−T_{S})]*[1+S2*(T_{S}−T_{R})], or
F(T))=F_{RT}*[1+S1*(T−T_{S})]/FC,
wherein F(T) is fluorescence of a reporter group at a temperature (T), F_{RT} is fluorescence at room temperature, SQ1 is a coefficient relating to the magnitude of the quadratic relationship between temperature and fluorescence, SQ2 is a coefficient relating to the magnitude of the linear relationship between temperature and fluorescence, T_{R} is room temperature, S1 is temperature sensitivity around a skin or body temperature region (T_{S}), S2 is a sensitivity applying to a region between room temperature (T_{R}) and skin temperature, and FC is a correction factor which accounts for T-skin, T-body, and T-room.

Thus, the present invention also encompasses methods of correcting fluorescence information emitted from a sensor, wherein corrected fluorescence is determined by any of the following formulae:
F(T)=F_{RT}*[1+SQ1*(T−T_{R})²+SQ2*(T−T_{R})],
F(T)=F_{RT}*[1+S1*(T−T_{S})]*[1+S2*(T_{S}−T_{R})], or
F(T))=F_{RT}*[+S1*(T−T_{S})]/FC,
wherein F(T) is fluorescence of a reporter group at a temperature (T), F_{RT} is fluorescence at room temperature, SQ1 is a coefficient relating to the magnitude of the quadratic relationship between temperature and fluorescence, SQ2 is a coefficient relating to the magnitude of the linear relationship between temperature and fluorescence, T_{R} is room temperature, S1 is temperature sensitivity around a skin or body temperature region (T_{S}), S2 is a sensitivity applying to a region between room temperature (T_{R}) and skin temperature, and FC is a correction factor which accounts for T-skin, T-body, and T-room. Such methods include measuring and/or determining any of the above variables and solving for F(T) and/or F_{RT} using one or more of the above formulae.

Further encompassed by the present invention are methods, which include converting a reference fluorescence of a reporter group at a reference temperature (T) into a fluorescence at a reference body temperature (such as a reference temperature of biological sample), for example, by the methods set forth above. Thereafter, the measured fluorescence may be compared to the transformed reference fluorescence. This is useful because fluorescence intensity measurements are relative measurements, not absolute. Measured fluorescence is compared to a reference fluorescence so the measured fluorescence or the reference fluorescence may be transformed. Further, the transformation of either measured fluorescence or reference fluorescence may include other factors besides temperature (to account for changes to background, etc).

The present invention also encompasses methods that include using temperature information from a biological sample in proximity to at least one binding protein having at least one reporter group attached thereto, to correct an initially determined concentration of at least one analyte in the biological sample. According to certain embodiments, the initially-determined concentration of at least one analyte is determined by the at least one reporter group providing a signal when the at least one binding protein binds to the at least one analyte.

The methods of the present invention can be extended to incorporate fluorescence resonance energy transfer (FRET) measurements, fluorescence polarization and fluorescence anisotropy. For example, according to such methods, measurements at different temperatures may be taken, a temperature-to-signal relationship may be determined, and appropriate corrections factors determined based on whether FRET measurements, fluorescence polarization or fluorescence anisotropy is being used.

According to certain embodiments, methods in accordance with the present invention include receiving luminescence information (such as intensity, wavelength of maximum emission, lifetime, FRET efficiency, polarization, etc.), receiving temperature information, and determining a corrected luminescence value based on the temperature information. According to these embodiments, the luminescence information may be received from at least one binding protein having at least one reporter group attached thereto, where the at least one reporter group luminesces upon protein-binding to at least one analyte. The temperature information may include information regarding the temperature of a biological sample in proximity to the at least one binding protein. Methods according to these embodiments may further include determining a corrected analyte concentration based on the corrected luminescence value.

Systems

Further included are systems that include the following: at least one binding protein having at least one reporter group attached thereto, where the at least one binding protein is capable of generating a signal upon binding of at least one analyte to the at least one binding protein; means for measuring the signal; means for measuring temperature of a biological sample in proximity to the at least one binding protein; and means for correcting the measured signal based on the measured temperature.

According to the systems of the present invention, means for measuring the signal include for example, any means for detecting a luminescent signal, be it intensity, lifetime, polarity, etc. as would be apparent to those skilled in the art. By way of non-limiting example, such means may include one or more detectors, such as a fluorometer. The fluorometer may be capable of measuring fluorescence emitted from the at least one reporter group upon binding of the at least one analyte to the at least one binding protein.

Any means for measuring temperature of a biological sample in proximity to the at least one binding protein may be used in accordance with the present invention. For example, means for measuring temperature may include a thermometer, such as a thermocouple, infrared device or other means known to those skilled in the art.

Any means for correcting the measured signal based on the measured temperature may be employed in accordance with the present invention. For example, the measured signal may be corrected by a computer, processor or other means.

Thus, according to certain embodiments, the present invention includes systems that include the following: at least one binding protein having at least one reporter group attached thereto; a fluorometer; a thermometer; and a processor. The luminescent signal from the reporter group changes in response to changing concentrations of at least one analyte to be detected. The processor may handle signal processing, mathematical manipulation of one or more signals, and/or data storage and handling. The computer or processor may be in physical contact with the other components of the optical system or, in other embodiments, may be physically separated by up to several meters from the other components of the optical system. In these embodiments, information from energy detectors and/or electronic processing elements in the optical system are communicated wirelessly to the computer or processor. The computer or processor may also store calibration information specific to the sensing element.

Other systems in accordance with the present invention may include at least one binding protein having at least one reporter group attached thereto, where the at least one binding protein is capable of generating a signal upon ligand binding; a fluorometer for measuring the signal; a thermometer capable of measuring temperature of a biological sample in proximity to the at least one binding protein; and a processor for correcting a measured luminescence based on the measured temperature.

Further systems in accordance with the present invention include a sensor capable of measuring or detecting concentration of at least one analyte in a biological sample and capable of measuring temperature of the biological sample, and a processor.

According to certain embodiments of the invention, the processor is adapted to be capable of providing a corrected analyte concentration based on the measured temperature.

Apparatuses Including a Memory and a Processor

Further embodiments of the present invention include apparatuses that include a memory for storing luminescence information of a reporter group and temperature information; and a processor for correcting luminescence information based on temperature information. Suitable forms of memory and suitable processors would be apparent to those skilled in the art.

According to certain embodiments, the processor is capable of determining a luminescence (L_{RT}) corrected to room temperature based on at least one of the following variables: luminescence L(T) of a reporter group at a temperature (T); room temperature (T_{R}); skin temperature (T_{S}); temperature sensitivity (S1) around a skin or body temperature region; and sensitivity (S2) applying to a region between room temperature (T_{R}) and skin temperature (T_{S}). Thus, certain processors in accordance with the present invention may be capable of determining a fluorescence (F_{RT}) corrected to room temperature based on at least one of the following: fluorescence F(T) of a reporter group at a temperature (T); room temperature (T_{R}); skin temperature (T_{S}); temperature sensitivity (S1) around a skin or body temperature region; and sensitivity (S2) applying to a region between room temperature (T_{R}) and skin temperature (T_{S}).

Programs

The present invention is also directed to programs adapted to cause a computer to execute the methods set forth herein and/or one or more portions thereof. Suitable programs can be prepared by those skilled in the art in view of the present disclosure.

Computer Readable Storage Mediums

The present invention is also directed to a computer-readable storage medium (also referred to herein as “machine-readable medium”), on which is recorded a program adapted to cause a computer to execute the methods set forth herein and/or one or more portions thereof. Suitable forms of computer-readable storage medium would be apparent to those skilled in the art. By way of non-limiting example, disks, CDs and other forms of storage medium including temporary storage via random access memory (RAM) presently known or later developed may be used in accordance with the present invention.

Also encompassed by the present invention are machine-readable mediums that include instructions, execution of which by a machine determines a corrected luminescence value. According to certain embodiments, the machine-readable instructions include a code segment for determining the luminescence (L_{RT}) value corrected to room temperature based on at least one of the following variables: luminescence L(T) of a reporter group at a temperature (T); temperature (T); room temperature (T_{R}); skin temperature (T_{S}); temperature sensitivity (S1) around a skin or body temperature region; and sensitivity (S2) applying to a region between room temperature (T_{R}) and skin temperature (T_{S}). According to certain embodiments, the machine-readable instructions include a code segment for determining the corrected luminescence value as a function of at least one of luminescence L(T) of a reporter group at temperature (T); temperature (T); and room temperature (T_{R}). According to other embodiments, the machine-readable instructions include a code segment for determining the corrected luminescence value as a function of at least one of luminescence L(T) of a reporter group at temperature (T); temperature (T); temperature sensitivity (S1) around a skin or body temperature region; and skin temperature (T_{S}).

Certain machine-readable medium in accordance with the present invention include instructions, execution of which by a machine determines a corrected fluorescence value. According to these embodiments, the machine-readable instructions include a code segment for determining a fluorescence (F_{RT}) corrected to room temperature based on at least one of the following: fluorescence F(T) of a reporter group at a temperature (T); temperature (T); room temperature (T_{R}); skin temperature (T_{S}); temperature sensitivity (S1) around a skin or body temperature region; and sensitivity (S2) applying to a region between room temperature (T_{R}) and skin temperature (T_{S}). According to certain embodiments, the machine-readable instructions include a code segment for determining the corrected fluorescence value as a function of at least one of fluorescence F(T) of a reporter group at temperature (T); temperature (T); and room temperature (T_{R}). According to other embodiments, the machine-readable instructions include a code segment for determining the corrected fluorescence value as a function of at least one of fluorescence F(T) of a reporter group at temperature (T); temperature (T); temperature sensitivity (S1) around a skin or body temperature region; and skin temperature (T_{S}).

Computer Data Signals

Further embodiments of the present invention include a computer data signal embodied in a transmission medium, where the computer data signal includes a computer-readable program code. According to certain embodiments, the program code includes a code segment for determining the luminescence (L_{RT}) value corrected to room temperature based on at least one of the following: luminescence L(T) of a reporter group at a temperature (T); temperature (T) room temperature (T_{R}); skin temperature (T_{S}); temperature sensitivity (S1) around a skin or body temperature region; and sensitivity (S2) applying to a region between room temperature (T_{R}) and skin temperature (T_{S}). According to certain embodiments, the program code includes a code segment for determining the corrected luminescence value as a function of at least one of luminescence L(T) of a reporter group at temperature (T); temperature (T); and room temperature (T_{R}). According to other embodiments, the program code includes a code segment for determining the corrected luminescence value as a function of at least one of luminescence L(T) of a reporter group at temperature (T); temperature (T); temperature sensitivity (S1) around a skin or body temperature region; and skin temperature (T_{S}).

Certain embodiments of the present invention include a computer data signal embodied in a transmission medium, where the computer data signal includes a computer-readable program code, where the program code includes a code segment for determining a fluorescence (F_{RT}) value corrected to room temperature. According to these embodiments, the fluorescence (F_{RT}) value corrected to room temperature may be based on at least one of the following: fluorescence F(T) of a reporter group at a temperature (T); temperature (T) room temperature (T_{R}); skin temperature (T_{S}); temperature sensitivity (S1) around a skin or body temperature region; and sensitivity (S2) applying to a region between room temperature (T_{R}) and skin temperature (T_{S}). According to certain embodiments, the program code includes a code segment for determining a corrected fluorescence value as a function of at least one of fluorescence F(T) of a reporter group at temperature (T); temperature (T); and room temperature (T_{R}). According to other embodiments, the program code includes a code segment for determining the corrected fluorescence value as a function of at least one of fluorescence F(T) of a reporter group at temperature (T); temperature (T); temperature sensitivity (S1) around a skin or body temperature region; and skin temperature (T_{S}).

While the present invention is satisfied by embodiments in many different forms, there will herein be described in detail embodiments of the invention, with the understanding that the present disclosure and examples are to be considered as exemplary and/or illustrative of the principles of the invention and are not intended to limit the scope of the invention to the embodiments illustrated and described. As would be apparent to skilled artisans, various changes and modifications are possible and are contemplated within the scope of the invention described, and may be made by persons skilled in the art without departure from the spirit of the invention.

### EXAMPLES

### Example 1

The objective of this experiment was to determine whether Continuous Glucose Monitoring System (“CGMS”) sensors are sensitive to temperature, and if so, to determine the magnitude and repeatability of the sensitivity. This experiment depicts, inter alia, calculations for correcting fluorescence if temperature is measured independently.

Experimental Methods

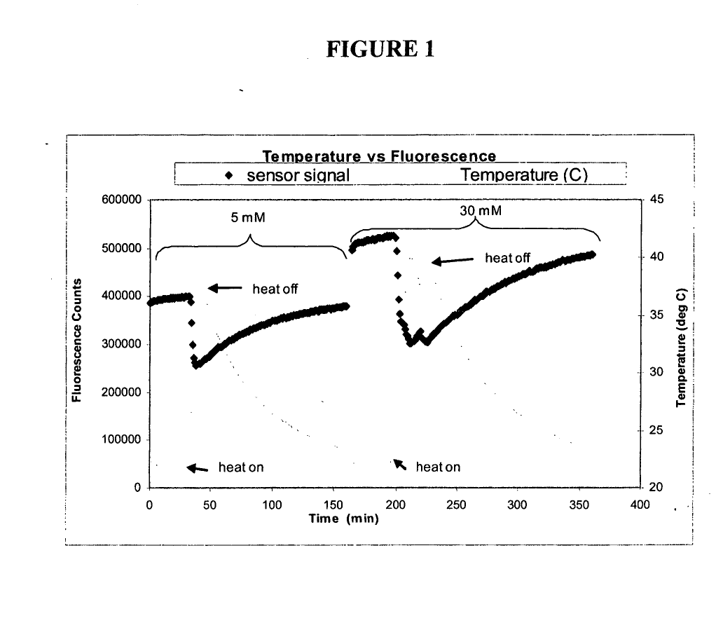

Several experiments were performed using various optical sensors (fiber-in-needle). Sensors were placed in scintillation vials containing glucose solutions (5 and 30 mM) and which were located in a heating block. The block temperature was cycled from room temperature to 40° C. and back to room temperature once for each glucose concentration. Sensor fluorescence and solution temperature data were collected. Sample data from one of those experiments are depicted in FIG. 1. Oscillations about the set point are due to the heating block controller.

Data Analysis

For each experiment, temperature differences were calculated (T—room temperature and T—pig skin reference temperature). Room temperature was assumed to be 21.3° C. and pig skin reference temperature was assumed to be 35° C. The fluorescence data from each sensor were divided into 4 segments:
all data for 5 mM,
all data for 30 mM,
all data for 5 mM and temp above 30° C., and
all data for 30 mM and temp above 30° C.

For each segment, slope and intercept were calculated using either the room temperature reference or the skin temperature reference (for above 30° C. segments).

Data were corrected for a 0.3% per hour constant drift associated with a 1 minute sample rate. It was noted during examination of early data sets that the temperature-fluorescence curve was not linear over the entire range 21-40° C. However, if only high temperature data were considered, the temperature-fluorescence relationship was more approximately linear.

Two correction factors were calculated. The first correction factor was to correct data to some skin reference temperature (T_{S}) and a second correction factor was used to correct data from skin reference temperature to room temperature. In practice, these two factors may be multiplied together. In the case of the sensors reported upon here, the temperature sensitivity about skin reference temperature, T_{S}, was −2.5%/° C., and the T_{S} to T_{R} correction factor used a sensitivity of −2.3%/° C.

A fluorescence measurement made at room temperature is designated “F_{RT},” and the same source at any arbitrary temperature is designated “F(T).” Then, assuming the relationship is substantially linear,
F(T)=F_{RT}+m*(T−T_{R}).

In the above formula, “m” has units of counts/degree C. and is negative. As indicated above, temperature sensitivity (S) is expressed as a fraction of the fluorescence signal lost (from the correct or reference signal) per degree.
S=m/F_{RT}, or m=S*F_{RT}

S has units of inverse temperature. S is expressed in terms of measured fluorescence, because the corrected signal F_{RT} is the unknown. Combining the previous two equations, we obtain:
F(T)=F_{RT}+m*(T−T_{R}), or
F(T)=F_{RT}+S*F_{RT}*(T−T_{R}), or
F(T)=F_{RT}*[1+S*(T−T_{R})], so that
F_{RT}=F(T)/[1+S*(T−T_{R})].

This equation provides a temperature-corrected fluorescence value. Assuming the temperature-fluorescence curve is substantially linear from room temperature to any in vivo temperature, then the equation above should serve. Recognizing the non-linearity in the data to be significant, a two-step approach may be taken. Assuming a sensitivity (S1) applying around the skin temperature (T_{S}) region and a sensitivity (S2) applying to the region between room temperature and skin temperature, then
F_{RT}=F(T)/[1+S1*(T−T_{S})]/[1+S2*(T_{S}−T_{R})].

Data thus far indicate that both S1 and S2 are negative and are approximately the same order of magnitude.

It may be conceptually easier to think of an in vivo temperature sensitivity (S1) and an in vitro to in vivo correction factor (FC):
FC=1/[1+S2*(T_{S}−T_{R})], so
F_{RT}=F(T)*FC/[1+S1*(T−T_{S})].

In this form, with typical values for T_{S}, T_{R}, and S2, FC is positive and greater than 1. In the case of the data reported upon here, FC=1.27.

It is also possible to fit a quadratic equation to the data:
F(T)=F_{RT}+a*(T−T_{R})²+b*(T−T_{R}), or
F(T)=F_{RT}+S1*F_{RT}*(T−T_{R})²+S2*F_{RT}*(T−T_{R}), or
F(T)=F_{RT}[1+S1*(T−T_{R})²+S2*(T−T_{R})], so that
F_{RT}=F(T)/[1+S1*(T−T_{R})²+S2*(T−T_{R})].

The quadratic correction should be slightly more accurate than the two-step approach, particularly for temperatures further from T_{S} or T_{R}. However, the sensitivity coefficients involved are not intuitively grasped, and the computation required is increased. For the data reported upon here, S1 is approximately +5e−2%/(° C.)² and S2 is approximately −3%/° C.

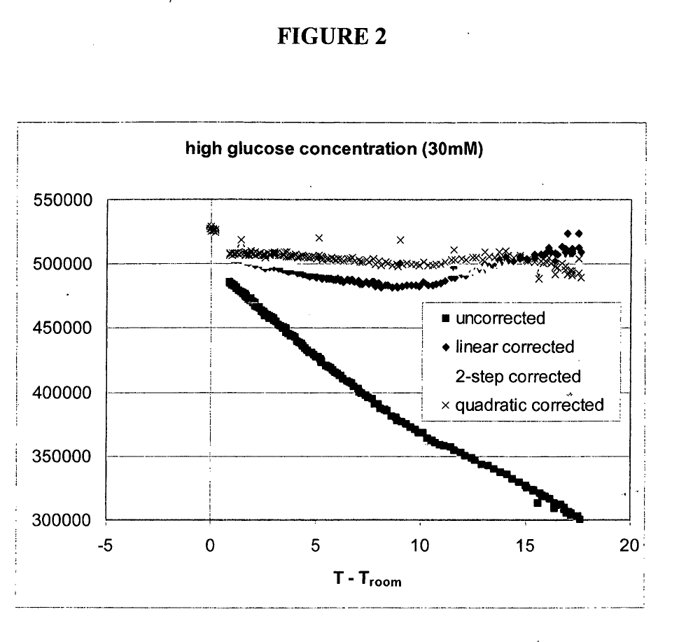

Fluorescence data from the high concentration (30 mM) part of the test are plotted as a function of temperature in FIG. 2 and data for the low glucose concentration (5 mM) part of the same test are plotted in FIG. 3. The F-T curves of uncorrected data are not linear in either case. The data were corrected back to 21° C. using sensitivities calculated from data assuming the following:
A linear relationship with 1 sensitivity for all temperatures,
A 2-sensitivity relationship assuming (S1 around T_{S} and S2 from T_{R} to T_{S}),
A quadratic relationship.

Note that for the 2-step approach the second sensitivity was only applied for temperatures greater than 30° C., so that below 30° C. the result is the same as the single sensitivity method. The 2-step method gives nearly the same correction as the quadratic method at high temperatures.

The corrections applied to the low and high glucose concentration data used the same sensitivities. The corrections are intended to change measured fluorescence back to a reference temperature (21° C.) equivalent. In the low glucose case of FIG. 3, the data collected at 21° C. lie on the same curve as all of the uncorrected data, and the corrected data at high temperatures is of the same magnitude. In the case of high glucose however, the data collected near 21° C. do not lie on the same curve as the rest of the data and were not used in calculation of temperature sensitivity. The corrected fluorescences at high temperatures are thus offset from the actual data near 21° C.

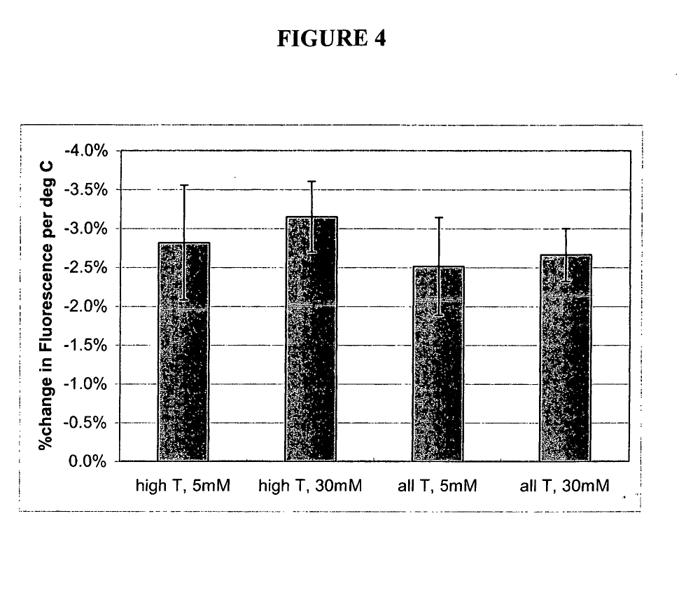

The overall and high-temperature sensitivities (using a linear fit) the sensor tests reported here are shown in FIG. 4. In particular, FIG. 4 depicts temperature sensitivities for a limited set of CGMS probes, and sensitivities may be different for other probes High temperature sensitivities were calculated using only data collected above 30° C. Percent change is calculated as the change in fluorescence per degree divided by the fluorescence at the reference temperature (35° C. for skin temp [“high T”], 21.3° C. for room temperature [“all T”]).

### Example 2

An example of temperature correction using a second luminescent reporter is presented below. In this example, both the sensing and reference reporting groups have temperature sensitivities largely quadratic in temperature, but the magnitude of sensitivity differs for each. In this example the reference reporting group is the same dye as used in the sensor, but is attached to a binding protein which is not sensitive to the presence of analyte over the range of expected analyte concentrations. In other embodiments, the reference dye could have different excitation/emission properties than the sensing group.

Experimental Methods

Experiments were performed using various optical sensors (fiber-in-needle) with one of two protein-dye combinations. Sensors were placed in scintillation vials containing 30 mM glucose solutions and which were located in a heating block. The block temperature was cycled from room temperature to 35° C. and back to room temperature twice. Sensor fluorescence and solution temperature data were collected.

Data Analysis

For the analyte-insensitive reporting group, the temperature sensitivity can be expressed as:
R(T)/R_{RT}=[1+5.2e−4*(T−T_{R})²−3.7e−2 (T−T_{R})],
where R(T) is the luminescence of a reference reporter group at a temperature (T), and R_{RT} is luminescence of a reference reporter group at room temperature (T_{R}).

The analyte-sensing reporting group temperature sensitivity can be expressed as:
F(T)/F_{RT}=[1+3e−4*(T−T_{R})²−3.4e−2 (T−T_{R})].

Additionally, at room temperature, the reference reporting group produced a relative fluorescence value of 12, while the sensing reporting group produced a relative fluorescence of 15. The methods described in this example are independent of the relative fluorescence intensities of the sensing and reference reporting groups.

The relationship between the sensing and reference fluorescence ratios can be well represented by a polynomial equation, arrived at by one of many means apparent to those skilled in the art, in this case
F(T)/F_{RT}=−0.2934−0.439*{R(T)/R_{RT}}²+1.74{R(T)/R_{RT}}, or
F_{RT}=F(T)/[−0.2934−0.439*{R(T)/R_{RT}}²+1.74{R(T)/R_{RT}}].

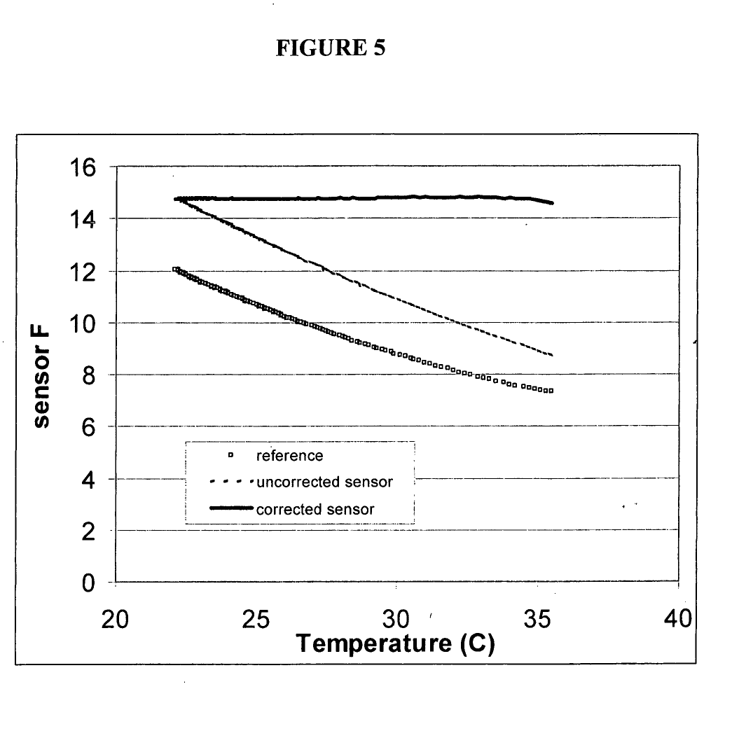

The sensing fluorescent signal was corrected via this equation and the reference signal ratio. The uncorrected and corrected sensor signals for one cooling cycle are shown in FIG. 5. The corrected signal is within +/−0.75% of the mean over the entire temperature range from 22° C. to 35° C.

The relationship between the sensing and reference signals could be expressed substantially linearly with only slight loss of final accuracy. Additionally, the temperature sensitivities could be compared over a narrower range, for example the range of expected skin temperatures, and a more accurate fit over that narrow range could be obtained. Also, the reference signal could be used to correct the sensor signal to a reference skin temperature, then the sensing-reporting group temperature sensitivity could be used to make the final transformation to room temperature. Alternatively, a reference fluorescence value at a reference temperature could be transformed to a signal at the reference skin temperature using the sensing-reporting group temperature sensitivity.

While the invention herein disclosed has been described by means of specific embodiments and applications thereof, numerous modifications and variations can be made thereto by those skilled in the art without departing from the scope of the invention as set forth in the claims.

1. A device comprising
at least one binding protein having at least one reporter group attached thereto; and
a thermometer.

2. The device of claim 1, wherein the at least one reporter group comprises a luminescent label.

3. The device of claim 1, wherein the thermometer is selected from the group consisting of a thermocouple and an infrared device.

4. The device of claim 1, wherein the reporter group is capable of providing a signal when the at least one binding protein binds to at least one analyte.

5. The device of claim 4, wherein the at least one analyte comprises at least one analyte selected from the group consisting of amino acids, peptides, polypeptides, proteins, carbohydrates, lipids, nucleotides, oligonucleotides, polynucleotides, glycoproteins or proteoglycans, lipoproteins, lipopolysaccharides, drugs, drug metabolites, small organic molecules, inorganic molecules, natural polymers and synthetic polymers.

6. The device of claim 4, wherein the at least one analyte comprises at least one analyte selected from the group consisting of glucose, galactose, lactate, fatty acids, c-reactive protein, carbohydrates and anti-inflammatory mediators.

7. A device comprising
an optical sensor capable of measuring at least one analyte in a biological sample in proximity to the optical sensor; and
a thermometer capable of measuring a temperature of said biological sample in proximity to the optical sensor.

8. A device comprising
at least one binding protein having at least one reporter group attached thereto;
wherein the at least one reporter group is capable of detecting a temperature of a biological sample in proximity to the at least one binding protein.

9. A method comprising
acquiring luminescence information of at least one reporter group;
acquiring temperature information of a biological sample; and
determining a corrected luminescence value based on the temperature information;
wherein the corrected luminescence value is indicative of a concentration of at least one analyte in the sample.

10. The method of claim 9, further comprising
determining a concentration of the at least one analyte based on the corrected luminescence value.

11. The method of claim 9, wherein the luminescence information comprises information from at least one binding protein having the at least one reporter group attached thereto, wherein the at least one reporter group luminesces when said at least one binding protein binds to the at least one analyte.

12. The method of claim 11, wherein the temperature information comprises information regarding the temperature of at least a portion of the biological sample in proximity to the at least one binding protein.

13. The method of claim 9, wherein the at least one analyte comprises at least one analyte selected from the group consisting of amino acids, peptides, polypeptides, proteins, carbohydrates, lipids, nucleotides, oligonucleotides, polynucleotides, glycoproteins or proteoglycans, lipoproteins, lipopolysaccharides, drugs, drug metabolites, small organic molecules, inorganic molecules, natural polymers and synthetic polymers.

14. The method of claim 9, wherein the at least one analyte comprises at least one analyte selected from the group consisting of glucose, galactose, lactate, fatty acids, c-reactive protein, carbohydrates and anti-inflammatory mediators.

15. The method of claim 9, wherein corrected luminescence is a function of at least one variable selected from the group consisting of
luminescence L(T) of the reporter group at temperature (T);
temperature (T); and
room temperature (T_{R}).

16. The method of claim 15, wherein corrected luminescence is determined using the following formula:
L_{RT}=L(T)/[1+SQ1*(T−T_{R})²+SQ2*(T−T_{R})]
wherein L_{RT} is luminescence at room temperature, L(T) is luminescence of the reporter group at a temperature (T), T_{R} is room temperature, SQ1 is a coefficient relating to the magnitude of the quadratic relationship between temperature and luminescence, and SQ2 is a coefficient relating to the magnitude of the linear relationship between temperature and luminescence.

17. The method of claim 15, wherein corrected luminescence is corrected fluorescence.

18. The method of claim 9, wherein corrected luminescence is a function of at least one variable selected from the group consisting of
luminescence (L(T)) of the reporter group at temperature (T);
temperature (T);
temperature sensitivity (S1) around a skin or body temperature region (T_{S}); and
sensitivity (S2) in a region between room temperature (T_{R}) and skin temperature (T_{S}).

19. The method of claim 18, wherein corrected luminescence is determined by a method comprising using the following formula:
L_{RT}=L(T)/[1+S1*(T−T_{S})]/[1+S2*(T_{S}−T_{R})]
wherein L_{RT} is luminescence at room temperature.

20. The method of claim 18, wherein corrected luminescence is corrected fluorescence.

21. The method of claim 18, wherein corrected luminescence is determined by a method comprising using the following formula:
L_{RT}=L(T)*FC/[1+S1*(T−T_{S})]
wherein L_{RT} is luminescence at room temperature, and FC is a correction factor which accounts for the change in luminescence due to the difference in the nominal temperature of skin of a mammal for which the analyte concentration is being determined, an internal body temperature of the mammal, and room temperature.

22. A method comprising
using temperature information from a biological sample in proximity to at least one binding protein, said protein having at least one reporter group attached thereto, to correct an initially determined concentration of at least one analyte in the biological sample;
wherein the initially-determined concentration of the at least one analyte is determined by the at least one reporter group providing a signal when the at least one binding protein binds to the at least one analyte.

23. A method of correcting fluorescence information comprising:
measuring fluorescence of a reporter group at multiple temperatures;
determining a temperature-to-signal relationship; and
determining at least one correction factor.

24. A system comprising
at least one binding protein having at least one reporter group attached thereto;
a fluorometer;
a thermometer; and
a processor.

25. The system of claim 24, wherein
said at least one binding protein is capable of generating a signal upon binding of at least one analyte to the at least one binding protein;
the fluorometer is capable of measuring the signal;
the thermometer is capable of measuring temperature of a biological sample in proximity to the at least one binding protein; and
the processor is capable of correcting a measured luminescence based on the measured temperature.

26. A system comprising
a sensor capable of detecting concentration of at least one analyte in a biological sample and capable of measuring temperature of the biological sample; and
a processor.

27. The system of claim 26, wherein the processor is adapted to be capable of providing a corrected analyte concentration based on the measured temperature.

28. An apparatus comprising
a memory for storing luminescence information of a reporter group and temperature information; and
a processor for correcting luminescence information based on temperature information.

29. The apparatus of claim 28, wherein the processor determines a corrected luminescence as a function of at least one variable selected from the group consisting of:
luminescence (L(T)) of a reporter group at temperature (T);
temperature (T);
room temperature (T_{R});
skin temperature (T_{S});
temperature sensitivity (S1) around a skin or body temperature region; and
sensitivity (S2) at a region between room temperature (T_{R}) and skin temperature (T_{S}).

30. A program, adapted to cause a computer to execute the method of claim 9.

31. A computer-readable storage medium, on which is recorded a program adapted to cause a computer to execute the method of claim 9.

32. A machine-readable medium including instructions, execution of which by a machine determines a corrected luminescence value, the machine-readable instructions comprising: a code segment for determining the corrected luminescence value as a function of at least one variable selected from the group consisting of:
luminescence (L(T)) of a reporter group at temperature (T);
temperature (T);
room temperature (T_{R});
skin temperature (T_{S});
temperature sensitivity (S1) around a skin or body temperature region; and
sensitivity (S2) at a region between room temperature (T_{R}) and skin temperature (T_{S}).

33. A computer data signal embodied in a transmission medium, comprising:
a computer-readable program code, said program code comprising a code segment for determining the corrected luminescence value as a function of at least one variable selected from the group consisting of:
luminescence (L(T)) of a reporter group at temperature (T);
temperature (T);
room temperature (T_{R});
skin temperature (T_{S});
temperature sensitivity (S1) around a skin or body temperature region; and
sensitivity (S2) at a region between room temperature (T_{R}) and skin temperature (T_{S}).
